(19) United States
(12) Patent Application Publication
(10) Pub. No.: US 2021/0161955 A2
(43) Pub. Date: Jun. 3, 2021

(54) COMPOSITIONS CONTAINING A CELL PRODUCT COMPRISING AN EXPANDED AND ENRICHED POPULATION OF SUPERACTIVATED CYTOKINE KILLER T CELLS AND METHODS FOR MAKING SAME

(71) Applicants: Jian XU, Jin Shan District, Shanghai (CN); Xiao ZHANG, Jin Shan District, Shanghai (CN); Jing WANG, Jin Shan District, Shanghai (CN); Ling ZHU, Jin Shan District, Shanghai (CN); Beverly LUBIT, Kinnelon, NJ (US)

(72) Inventors: Jian XU, Shanghai (CN); Xiao ZHANG, Shanghai (CN); Jing WANG, Shanghai (CN); Ling ZHU, Shanghai (CN); Beverly LUBIT, Kinnelon, NJ (US)

(21) Appl. No.: 16/682,422

(22) Filed: Nov. 13, 2019

Related U.S. Application Data
(60) Provisional application No. 62/760,077, filed on Nov. 13, 2018.

(51) Int. Cl. A61K 35/17; C12N 5/00; C12N 5/0783; A61P 35/00

(52) U.S. Cl. CPC A61K 35/17; C12N 5/0018; C12N 5/0646; C12N 5/0638; A61P 35/00; C12N 2501/2302; C12N 2501/515; C12N 2501/2315; C12N 2501/2312; C12N 2501/599; C12N 2500/36; C12N 2502/1121; C12N 2501/2307

(57) ABSTRACT

The present disclosure describes a pharmaceutical composition comprising a pharmaceutically acceptable carrier and a cell product comprising an expanded and enriched population of superactivated cytokine killer T cells, and methods for manufacturing the cell product.

### RELATED APPLICATIONS

This application claims the benefit of and priority to U.S. Provisional Application No. 62/760,077, filed Nov. 13, 2018, the contents of which is expressly incorporated herein by reference in its entirety.

### BACKGROUND

Lymphocytes are a type of white blood cell involved in immune system regulation. Lymphocytes are much more common in the lymphatic system, and include B cells, T cells, killer T-cells, and natural killer (NK) cells. There are two broad categories of lymphocytes, namely T cells and B cells. T-cells are responsible for cell-mediated immunity whereas B-cells are responsible for humoral immunity (relating to antibodies). T-cells are so-named such because these lymphocytes mature in the thymus; B-cells mature in bone marrow. B cells make antibodies that bind to pathogens to enable their destruction. CD4+(helper) T cells co-ordinate the immune response. CD8+(cytotoxic) T cells and Natural Killer (NK) cells are able to kill cells of the body that are, e.g., infected by a virus or display an antigenic sequence.

The immune response to invading pathogens requires the successful activation of innate immunity, which informs the development of the subsequent adaptive immune response.

Natural killer T (NKT) cells are a heterogeneous subset of specialized T cells (Brennan et al., Nat Rev Immunol. 2013 February; 13(2):101-17). These cells exhibit an innate cell-like feature of quick response to antigenic exposure in combination with adaptive cell's precision of antigenic recognition and diverse effector responses (Salio et al., Annu Rev Immunol. 2014; 32( ):323-66). Like conventional T cells, NKT cells undergo thymic development and selection and possess T cell receptor (TCR) to recognize antigens (Berzins et al., Immunol Cell Biol. 2004 June; 82(3):269-75).

Diversity of the TCR gene is generated by rearrangement of the V and J gene segments during T cell development in the thymus. (Makino, Y., et al (1993) J. Exptl Med. 177: 1399-1408). The TCR V and J gene segments, like Ig genes, possess recombination signals in which heptamer and nonamer sequences, separated by a 12/23 bp spacer, are flanked by germline V and J gene segments. Id.

Natural killer T (NKT) cells represent a small population of T lymphocytes defined by the expression of both αβ T-cell receptors (TCR) and some lineage markers of NK cells. However, unlike conventional T cells, TCR expressed by NKT cells recognize lipid antigens presented by the conserved and non-polymorphic MHC class 1 like molecule CD1d (Godfrey et al., Nat Immunol. 2015 November; 16(11):1114-23). In addition to TCRs, NKT cells also possess receptors for cytokines such as IL-12, IL-18, IL-25, and IL-23 similar to innate cells such as NK and innate lymphoid cells (Cohen et al., Nat Immunol. 2013 January; 14(1):90-9). These cytokine receptors can be activated by steady state expression of these inflammatory cytokines even in the absence of TCR signals. Thus, NKT cells can amalgamate signals from both TCR-mediated stimulations and inflammatory cytokines to manifest prompt release of an array of cytokines (Kohlgruber et al., Immunogenetics. 2016 August; 68(8):649-63). These cytokines can in turn modulate different immune cells present in the tumor microenvironment (TME) thus influencing host immune responses to cancer.

As shown in Table 1, there are a number of subtypes of NKT cells, which can be determined through their T cell receptor (TCR) usage, cytokine production, expression of specific surface molecules and reactivity.

TABLE 1

| NKT Cell | | | |
|---|---|---|---|
| Subset | | Mouse | Human |
| Type I | TCR | Vα14-Jα18; Vβ8.2/7/2 | Vα24-Jα18; Vβ11 |
| | Subsets | CD4+, DN | CD4+, CD8+, DN |
| | Ligand | αGalCer | αGalCer |
| | Restriction | CD1d | CD1d |
| | NK | NK1.1+/− | CD161+/− |
| | Receptors | | |
| Type II | TCR | Vα3.2-Jα9 or Vα8; Vβ8 | Diverse |
| | Subsets | CD4+, DN | CD4+, CD8+ |
| | Ligand | Sulfatide, lysosulfatide, | Sulfatide, lysosulfatide, |
| | | lysophosphatidylcholine | lysophosphatidylcholine |
| | Restriction | CD1d | CD1d |
| | NK | NK1.1+/− | CD161+ |
| | Receptors | | |

### Type-I NKT Cells

Broadly, CD1d-restricted NKT cells can be divided into two main subsets based on their TCR diversity and antigen specificities. The most extensively characterized subtype of NKT cells are the type-I or invariant natural killer T cell (iNKT cells) (Matsuda et al, Curr Opin Immunol, 20: 358-68, 2008). Type-I (invariant) NKT cells (iNKT cells), so named because of their limited TCR repertoire, express a semi-invariant TCR (iTCR) α chain (Vα14-Jα18 in mice, Vα24-Jα18 in humans) paired with a heterogeneous Vβ chain repertoire (V β 2,7 or 8.2 in mice and V β 11 in humans) (Brennan et al., Nat Rev Immunol. 2013 February; 13(2):101-17; Salio et al., Annu Rev Immunol. 2014; 320:323-66). The prototypic antigen for type-I NKT cells is galactosylceramide (α-GalCer or KRN 7000), which was isolated from a marine sponge as part of an antitumor screen (Kawano et al., Science. 1997 Nov. 28; 278(5343):1626-9). α-GalCer is a potent activator of type-I NKT cells, inducing them to release large amounts of interferon-γ (IFN-γ), which helps activate both CD8+ T cells and antigen presenting cells (APCs) (Kronenberg, Nat Rev Immunol. 2002 August; 2(8):557-68). The primary techniques used to study type-I NKT cells include staining and identification of type-I NKT cells using CD1d-loaded α-GalCer tetramers, administering α-GalCer to activate and study the functions of type-I NKT cells, and finally using CD1d deficient mice (that lack both type-I and type-II NKT) or Jα18-deficient mice (lacking only type-I NKT) (Berzins et al., Immunol Cell Biol. 2004 June; 82(3):269-75). It has been reported that Jα18-deficient mice in addition to having deletion in the Traj18 gene segment (essential for type-I NKT cell development), also exhibited an overall lower TCR repertoire caused by influence of the transgene on rearrangements of several Ja segments upstream Traj18, complicating interpretations of data obtained from the Jα18-deficient mice (Bedel et al., Nat Immunol. 2012 Jul. 19; 13(8):705-6). To overcome this drawback, a new strain of Jα18-deficient mice lacking type-I NKT cells while maintaining the overall TCR repertoire has been generated to facilitate future studies on type-I NKT cells (Chandra et al., Nat Immunol. 2015 August; 16(8):799-80). Type-I NKT cells can be further subdivided based on the surface expression of CD4 and CD8 into CD4+ and CD4−CD8− (double-negative, or DN) subsets and a small fraction of CD8+ cells found in humans (Bendelac et al., Science. 1994 Mar. 25; 263(5154):1774-8; Lee et al., J Exp Med. 2002 Mar. 4; 195(5):637-41). Type-I NKT cells are present in different tissues in both mice and humans, but at higher frequency in mice (Arrenberg et al., J Cell Physiol. 2009 February; 218(2):246-50).

Type-I NKT cells possess dual reactivity to both self and foreign lipids. Even at steady state, type-I NKT cell have an activated/memory phenotype (Bendelac et al., Annu Rev Immunol. 2007; 25( ):297-336; Godfrey et al., Nat Immunol. 2010 March; 11(3):197-206).

Functionally distinct subsets of NKT cells analogous to Th1, Th2, Th17, and TFH subsets of conventional T cells have been described. These subsets express the corresponding cytokines, transcription factors and surface markers of their conventional T cell counterparts (Lee et al., Immunity. 2015 Sep. 15; 43(3):566-78). Type-I NKT cells have a unique developmental program that is regulated by a number of transcription factors (Das et al., Immunol Rev. 2010 November; 238(1):195-215). Transcriptionally, one of the key regulators of type-I NKT cell development and activated memory phenotype is the transcription factor promyelocytic leukemia zinc finger (PLZF). In fact, PLZF deficient mice show profound deficiency of type-I NKT cells and cytokine production (Kovalovsky D, et al., Nat Immunol (2008) 9:1055-64.10.1038/ni.164; Savage A K et al., Immunity (2008) 29:391-403). Other transcription factors that are known to impact type-I NKT cell differentiation are c-Myc (Dose et al., Proc Natl Acad Sci USA. 2009 May 26; 106(21):8641-6), RORγt (Michel et al., Proc Natl Acad Sci USA. 2008 Dec. 16; 105(50):19845-50), c-Myb (Hu et al., Nat Immunol. 2010 May; 11(5):435-41), Elf-1 (Choi et al., Blood. 2011 Feb. 10; 117(6):1880-7), and Runx1 (Egawa et al., Immunity. 2005 June; 22(6):705-16). Furthermore, transcription factors that control conventional T cell differentiation, such as Th1 lineage specific transcription factor T-bet and Th2 specific transcription factor GATA-3, can also affect type-I NKT cell development (Kim et al., J Immunol. 2006 Nov. 15; 177(10):6650-9; Townsend et al., Immunity. 2004 April; 20(4):477-94). Aside from transcription factors, SLAM-associated protein (SAP) signaling pathway can also selectively control expansion and differentiation of type-I NKT cells (Nichols et al., Nat Med. 2005 March; 11(3):340-5). Type-I NKT cells have been shown to respond to both self and foreign α and β linked glycosphingolipids (GSL), ceramides, and phospholipids (Macho-Fernandez et al., Front Immunol. 2015; 6: 362). Type-I NKT cells have been reported to mostly aid in mounting an effective immune response against tumors (McEwen-Smith et al., Cancer Immunol Res. 2015 May; 3(5):425-35; Robertson et al., Front Immunol. 2014; 5( ):543; Ambrosino et al., J Immunol. 2007 Oct. 15; 179(8):5126-36).

### Type-II NKT Cells

Type-II NKT cells, also called diverse or variant NKT cells, are CD1d-restricted T cells that express more diverse alpha-beta TCRs and do not recognize α-GalCer (Cardell et al., J Exp Med. 1995 Oct. 1; 182(4):993-1004). Type-II NKT cells are a major subset in humans with higher frequency compared to type-I NKT cells. Due to an absence of specific markers and agonistic antigens to identify all type-II NKT cells, characterization of these cells has been challenging. Different methodologies employed to characterize type-II NKT cells include, comparing immune responses between Jα18−/−(lacking only type-I NKT) and CD1d−/−(lacking both type I and type-II NKT) mice, using 24 αβ TCR transgenic mice (that overexpress Vα3.2/Vβ9 TCR from type-II NKT cell hybridoma VIII24), using a Jα18-deficient IL-4 reporter mouse model, staining with antigen-loaded CD1d tetramer and assessing binding to type-II NKT hybridomas [reviewed in Macho-Fernandez, Front Immunol. 2015; 6:362)].

The first major antigen identified for self-glycolipid reactive type-II NKT cells in mice was myelin derived glycolipid sulfatide (Arrenberg et al., J Cell Physiol. 2009 February; 218(2):246-50; Jahng et al., J Exp Med. 2001 Dec. 17; 194(12):1789-99). Subsequently, sulfatide and lysosulfatide reactive CD1d-restricted human type-II NKT cells have been reported ((Shamshiev et al., J. Exp. Med. 2002; 195:1013-1021; Blomqvist et al., Eur J Immunol. 2009 July; 39(7): 1726-1735)). Sulfatide specific type-II NKT cells predominantly exhibit an oligoclonal TCR repertoire (V a 3/V a 1-J a 7/J a 9 and V β 8.1/V β 3.14 β 2.7) (Arrenberg et al., J Cell Physiol. 2009 February; 218(2):246-50). Other self-glycolipids such as β GlcCer and β GalCer have been shown to activate murine type-II NKT cells (Rhost et al., Scand J Immunol. 2012 September; 76(3):246-55; Nair et al., Blood. 2015 Feb. 19; 125(8):1256-71). It was reported that two major sphingolipids accumulated in Gaucher disease (GD), β-glucosylceramide (β GlcCer) and its deacylated product glucosylsphingosine, are recognized by murine and human type-II NKT cells (Nair et al., Blood. 2015 Feb. 19; 125(8):1256-71). In an earlier study, it was shown that lysophosphatidylcholine (LPC), lysophospholipid markedly upregulated in myeloma patients was an antigen for human type-II NKT cells (Chang et al., Blood. 2008 Aug. 15; 112(4):1308-16).

Type-II NKT cells can be distinguished from type-I NKT cells by their predominance in humans versus mice, TCR binding and distinct antigen specificities (J Immunol. 2017 Feb. 1; 198(3):1015-1021).

Crystal structures of type-II NKT TCR-sulfatide/CD1d complex and type-I NKT TCR-α-GalCer/CD1d complex provided insights into the mechanisms by which NKT TCRs recognize antigen (Girardi et al., Immunol Rev. 2012 November; 250(1):167-79). The type-I NKT TCR was found to bind α-GalCer/CD1d complex in a rigid, parallel configuration mainly involving the α-chain. The key residues within the CDR2β, CDR3α, and CDR1α loops of the semi-iTCR of type-I NKT cells were determined to be involved in the detection of the α-GalCer/CD1d complex (Pellicci et al, Immunity. 2009 Jul. 17; 31(1):47-59). On the other hand, type-II NKT TCRs contact their ligands primarily via their CDR3β loop rather than CDR3 α loops in an antiparallel fashion very similar to binding observed in some of the conventional MHC-restricted T cells (Griardi et al., Nat Immunol. 2012 September; 13(9):851-6). Ternary structure of sulfatide-reactive TCR molecules revealed that CDR3 α loop primarily contacted CD1d and the CDR3β determined the specificity of sulfatide antigen (Patel et al., Nat Immunol. 2012 September; 13(9):857-63). The flexibility in binding of type-II NKT TCR to its antigens akin to TCR-peptide-MHC complex resonates with its greater TCR diversity and ability to respond to wide range of ligands.

However, despite striking differences between the two subsets, similarities among the two subsets have also been reported. For example, both type-I and type-II NKT cells are autoreactive and depend on the transcriptional regulators PLZF and SAP for their development (Rhost et al., Scand J Immunol. 2012 September; 76(3):246-55). Although, many type-II NKT cells seem to have activated/memory phenotype like type-I NKT cells, in other studies, a subset of type-II NKT cells also displayed naïve T cell phenotype (CD45RA+, CD45RO−, CD62high, and CD69−/low) (Arrenberg et al., Proc Natl Acad Sci USA. 2010 Jun. 15; 107(24):10984-9). Type-II NKT cells are activated mainly by TCR signaling following recognition of lipid/CD1d complex (Roy et al., J Immunol. 2008 Mar. 1; 180(5):2942-50) independent of either TLR signaling or presence of IL-12 (Zeissig et al., Ann N Y Acad Sci. 2012 February; 1250:14-24).

### T Cell Development

As T cells develop in the thymus, TCR signals provide critical checkpoints as cells transit through the various stages of maturation. (See Huang, E. Y., et al, J. Immunol. (2003) 171: 2296-2304). For example, a pre-TCR signal is necessary for the most immature thymocyte subset, termed double negative (DN), to develop into double-positive (DP) thymocytes, expressing both CD4 and CD8. Id. The assembly and surface expression of CD3, pre Ta, and a functionally rearranged TCRβ-chain mediate this checkpoint, termed β selection. Id. After successful pre-TCR signaling, DN thymocytes undergo many rounds of division and multiple phenotypic changes. Id. In addition to genes that encode pre-TCR components, a number of other genes, which either affect pre-TCR signaling indirectly or are required for the numerous cellular changes seen during the DN to DP transition, regulate maturation. Id.

### Type-I NKT Cell Development

In both mice and humans, Type-I NKT cells segregate from conventional T cells during development at the double-positive (CD4+CD8+, DP) thymocyte stage, coincident with TCR αβ expression (Godfrey D I, Berzins S P Nat Rev Immunol. 2007 July; 7(7):505-18). Generation of the canonical TCRα used by type-I NKT cells is widely believed to be a random event, for although the amino acids which define the invariant Vα14-Jα18 rearrangement never vary, sequencing analysis has revealed that the nucleotides used to code for these amino acids are diverse (Lantz O, Bendelac A J Exp Med. 1994 Sep. 1; 180(3):1097-106). Due to structural constraints on recombination events in the TCRα locus, the numerous Vα and Jα gene segments become accessible for recombination as a function of their relative location in the locus. As a result, the Vα 14 gene segment only starts rearranging with Jα18 within a 24-48 h window before birth (Hager E. et al. J Immunol. 2007 Aug. 15; 179(4):2228-34). This explains the relatively late appearance of NKT cells in the thymus and is consistent with random generation of the canonical Vα14-Jα18 rearrangement within a common T cell progenitor pool. Furthermore, the frequency of the earliest identified NKT cell precursor was estimated to be 1 cell per 10⁶ thymocytes (Benlagha K. et al. J Exp Med. 2005 Aug. 15; 202(4):485-92). Together, these data support the notion that Vα14-Jα18 rearrangement occurs randomly at very low frequency.

As with conventional T cells, type-I NKT cell development requires recognition of self. The restriction element CD1d is expressed by both DP thymocytes and epithelial cells in the thymus. However, early studies revealed that type-I NKT cells are selected at the DP stage by CD1d-expressing DP cells themselves as opposed to epithelial cells that drive the selection of conventional T cells. Such a mode of selection was hypothesized to impart the unique developmental program of type-I NKT cells to the selected thymocytes. Recently, it was demonstrated that homotypic interactions across the DP-DP synapse generated “second signals” that are mediated by the cooperative engagement of the homophilic receptors of at least two members of the signaling lymphocytic-activation molecule (SLAM) family (Slamf1 [SLAM] and Slamf6 [Ly108]) [8λλ-10λλ]. Such engagements lead to the downstream recruitment of the adaptor SLAM-associated protein (SAP) and the Src kinase Fyn, which were previously recognized as essential for the expansion and differentiation of the type-I NKT cell lineage (Godfrey D I, 2007).

Once type-I NKT cells have been positively selected, they expand in the thymus and undergo an orchestrated maturation process that ultimately leads to the acquisition of their activated NK-like phenotype. This process relies on the proper expression of cytokine receptors, signal transduction molecules (e.g. Fyn, SAP), transcription factors (e.g. NFκB, T-bet, Ets1, Runx1, RORγ, Itk, Rlk, AP-1) (see Godfrey D I, 2007 for reviews), and co-stimulatory molecules such as CD28 and ICOS (Hayakawa et al., J Immunol. 2001 May 15; 166(10):6012-8; Akbari et al., J Immunol. 2008 Apr. 15; 180(8): 5448-5456). Most type-I NKT cells leave the thymus in an immature stage (as defined by the absence of expression of NK receptors such as NK1.1) and fulfill their terminal maturation in the periphery (Benlagha K. et al., Science. 2002 Apr. 19; 296(5567):553-5; McNab F W et al., J Immunol. 2005 Sep. 15; 175(6):3762-8). However, a sizeable fraction of these NK1.1-type-I NKT cells in the peripheral organs do not acquire expression of NK markers and in fact represent mature cells that are functionally distinct from their NK1.1+ thymic counterpart (McNab et al., J Immunol. 2007; 179:6630-6637).

The egress of type-I NKT cells from the thymus to the periphery requires lymphotoxin (LT) αβ signaling through the LTβ receptor expressed by thymic stromal cells (Franki A S et al., Proc Natl Acad Sci USA. 2006 Jun. 13; 103(24):9160-5). Such signaling in turn regulates thymic medullary chemokine secretion (Zhu M. et al., J Immunol. 2007 Dec. 15; 179(12):8069-75). Establishment of type-I NKT cells tissue residency in the periphery requires expression of the Sphingosine1-Phosphate 1 receptor (S1P1R) by type-I NKT cells (Allende M L et al., FASEB J. 2008 January; 22(1):307-15) and more specifically expression of CxCR6 for liver localization (Geissmann F. et al., PLoS Biol. 2005 April; 3(4):e113).

However, many type-I NKT cells remain in the thymus, mature to the NK1.1+ phenotype there, and become long-lived residents (Berzins S P et al. J Immunol. 2006 Apr. 1; 176(7):4059-65). The mechanisms responsible for the export/retention of type-I NKT cells from the thymus at various developmental stages are unknown.

### Type-I NKT Cell Activity

Type-I NKT cells have been shown to have many different activities during an immune response. Not only do they have the capacity to rapidly and robustly produce cytokines and chemokines, they also have the ability, as their name would suggest, to kill other cells. In addition, they have been shown to influence the behavior of many other immune cells. In this section, the multitude of functional properties that have been attributed to type-I NKT cells is described.

Cytokine and Chemokine Production

Type-I NKT cells were originally identified as an unusual T cell population with NK markers that had the unique capacity to rapidly and robustly produce IL-4 upon the injection of anti-CD3 antibodies in mice. Later studies revealed that while this robust IL-4 production was a signature of Type-I NKT cells, it was not the only cytokine type-I NKT cells can produce. Type-I NKT cells have been shown to produce IFN-γ and IL-4, as well as IL-2, IL-5, IL-6, IL-10, IL-13, IL-17, IL-21, TNF-α, TGF-β and GM-CSF (Bendelac A. et al., Annu Rev Immunol. 2007; 25( ):297-336; Gumperz J E et al., J Exp Med. 2002 Mar. 4; 195(5):625-36). Type-I NKT cells are also known to produce an array of chemokines (Chang Y J et al., Proc Natl Acad Sci USA. 2007 Jun. 19; 104(25):10299-304).

The rapid and dual production of IL-4 and IFNγ by type-I NKT cells in vivo following administration of the α-GalCer antigen has become a trademark feature of type-I NKT cells. In fact, within 2 h of in vivo exposure to antigen, intracellular analysis of ex vivo type-I NKT cells from naïve mice revealed that the majority of type-I NKT cells in the liver produced both IL-4 and IFNγ (Matsuda J L et al., J Exp Med. 2000 Sep. 4; 192(5):741-54). How type-I NKT cells from unsensitized mice produce cytokines so rapidly when activated is unclear. However, the observation that resting type-I NKT cells have high levels of IL-4 and IFNγ mRNAs provides one potential mechanism (Matsuda J L et al., Proc Natl Acad Sci USA. 2003 Jul. 8; 100(14):8395-400; Stetson D B et al., J Exp Med. 2003 Oct. 6; 198(7):1069-76).

Type-I NKT cells also regulate their cytokine production at the transcriptional level. Several transcription factors known to regulate cytokine gene transcription in conventional T cells (T-bet, GATA-3, NFκB], c-Rel, NFAT, AP-1, STATs, Itk) have also been implicated in type-I NKT cells. For example, type-I NKT cells appear to co-express both T-bet and GATA-3 transcription factors leading to the transcription of both IFNγ and IL-4 mRNAs. This is in contrast to conventional T cells where T-bet has been shown to repress the expression of GATA-3 and vice versa.

Cytolytic Activity of Type-I NKT Cells

Type-I NKT cells express high levels of granzyme B, perforin, and FasL, consistent with a cytolytic function for these cells. In vitro assays have demonstrated that type-I NKT cells have the ability to kill antigen-pulsed APCs in a CD1d-dependent manner. In addition, several mouse models have revealed that type-I NKT cells play an important role in tumor surveillance and tumor rejection. In some tumor models, IFNγ production by type-I NKT cells is instrumental in the activation of NK cells, which in turn mount a robust anti-tumor response (Crowe N Y et al., J Exp Med. 2002 Jul. 1; 196(1):119-27). Similarly, type-I NKT cells have been shown to recognize and respond to bacterial antigens and participate in bacterial clearance (Mattner et al., Nature. 2005 Mar. 24; 434(7032):525-9; Ranson et al., J Immunol. 2005 Jul. 15; 175(2):1137-44).

Regulation of Other Immune Cells

Early studies demonstrated that type-I NKT cell-derived cytokines can activate several other cell types, including NK cells, conventional CD4+ and CD8+ T cells, macrophages and B cells, and recruit myeloid dendritic cells (Kronenberg M, Gapin L Nat Rev Immunol. 2002 August; 2(8):557-68). Type-I NKT cells can also modulate the recruitment of neutrophils through their secretion of IFNγ (Nakamatsu M. et al., Microbes Infect. 2007 March; 9(3):364-74). Further, cross-talk between CD4+CD25+ regulatory T cells (Treg) and type-I NKT cells has been described, where activated type-I NKT cells quantitatively and qualitatively modulate Treg function through an IL-2 dependent mechanism, while Treg can suppress type-I NKT cell functions by cell-contact-dependent mechanisms (LaCava A. et al., Trends Immunol. 2006 July; 27(7):322-7). A similar cross-regulation between type-I NKT cells and other CD1d-restricted NKT cells that do not express the invariant TCR-α chain that characterize type-I NKT cells (type-II NKT cells), has also been observed (Ambrosino E. et al., J Immunol. 2007 Oct. 15; 179(8):5126-36). Type-I NKT cells have also been reported to synergize with γδ T cells in a model of allergic airway hyper-responsiveness (Jin N. et al., J Immunol. 2007 Sep. 1; 179(5):2961-8). Finally, it has been recognized for some time that systemic type-I NKT cell activation by α-GalCer injection induces activation of B cells non-specifically. Data show that purified type-I NKT cells from lupus-prone NZB/W F1 mice can spontaneously increase antibody secretion by B-1 and marginal zone B cells but not follicular zone B cells (Takahashi T, Strober S Eur J Immunol. 2008 January; 38(1):156-65). Direct interactions between type-I NKT cells and the B cell subsets were necessary and the effect could be blocked by anti-CD1d and anti-CD40L mAbs (Takahashi T, 2008). C57BL/6 mice immunized with proteins and α-GalCer developed antibody titers 1-2 logs higher than those induced by proteins alone and increased the frequency of memory B cells generated (Galli G et al., Proc Natl Acad Sci USA. 2007; 104:3984-3989). The mechanism was mediated through the combined action of CD40-CD40L interactions and cytokine secretion. CD1d expression by B cells is also required for the type-I NKT cell enhanced response, suggesting cognate interaction between type-I NKT cells and B cells (Lang G A et al., Blood. 2008 Feb. 15; 111(4):2158-62).

### Antigens Recognized by Type-I NKT Cells

The first described type-I NKT cell ligand was α-Galactosylceramide (α-GalCer), which was identified from a panel of marine extracts for its anti-tumor activity (Kawano T. et al., Science. 1997 Nov. 28; 278(5343):1626-9). Since then, many more type-I NKT cell antigens have been discovered, including both endogenous and exogenous antigens. Unlike conventional T cell antigens that are predominantly peptides presented by MHC molecules, type-I NKT cell antigens have a distinct lipid component to them. Most type-I NKT cell antigens defined to date share a common structure: a lipid tail that is buried into CD1d and a sugar head group that protrudes out of CD and makes contact with the NKT TCR. The main exception to this is the type-I NKT antigen phosphatidylethanolamine, which lacks a sugar head group.

Recognition of Antigens by NKT Cells

The unique antigen specificity of type-I NKT cells is dictated by the expression of the semi-invariant TCR. How this TCR, which was known to have a similar overall structure to known peptide/MHC reactive TCRs, might instead recognize glycolipid antigens in the context of CD1d was the subject of constant speculation. Crystallographic success and mutational analyses have exposed how this TCR recognizes CD1d/glycolipid complexes. The crystal structure of a human type-I NKT TCR in complex with CD1d/α-GalCer revealed a unique docking strategy that differed from known TCR/MHC/peptide interactions (Borg et al., Nature. 2007; 448:44-49). Compared with conventional TCR-MHC interactions, where TCR engages the distal portion of the MHC in a diagonal orientation, the type-I NKT TCR docked at the very end of, and parallel to, the CD1d-α-Galcer complex. In the structure, the binding surface between the type-I NKT TCR and CD1d-α-GalCer complex was composed primarily of three out of the six complementarity-determining region (CDR) loops: CDR1α, CDR3α and CDR2β, with the invariant TCRα chain dominating the interaction with both the glycolipid and CD1d, while the role of the TCRα chain was restricted to the CDR2β loop interacting with the α1 helix of CD1d. CDR3β, the only hypervariable region of the type-I NKT TCR, which usually mediates antigen specificity together with CDR3α for conventional TCR, did not make any contact with the antigen. Thus, recognition of α-Galcer-CD1d by the type-I NKT TCR is entirely mediated by germline-encoded surface on the type-INKT TCR.

These results were confirmed and extended through an extensive mutational analyses of both mouse and human type-I NKT TCRs (Browne et al., Nat Immunol. 2007; 8: 1105-1113). The results confirmed an energetic ‘hot-spot’ formed by residues within the CDR1α, CDR3α and CDR2β loops of the TCR that were critical for the recognition of the α-GalCer-CD1d complex and provided the basis for the extremely biased TCR repertoire of type-I NKT cells. In the mouse system, this ‘hot-spot’ was similarly required for recognition of structurally different glycolipid antigens such as α-GalCer and iGb3. Because recognition of diverse glycolipid antigens used the same germline-encoded residues, these observations suggest that the type-I NKT TCR functions as a pattern-recognition receptor (Browne et al., Nat Immunol. 2007; 8: 1105-1113). In this way, different NKT cell clones have overlapping antigen specificity despite diversity in the TCRβ chain.

### Activation of Type-I NKT Cells

Cognate Recognition and Activation of Type-I NKT Cells by Foreign Antigen

Microbial glycolipids presented as cognate antigens that activate type-I NKT cells have been identified. Type-I NKT cells have been shown to directly recognize α-linked glycosphingolipids and diacylglycerol antigens that are expressed by bacteria such as Sphingomonas, Ehrlichia and Borrelia burgdorferi in a CD1d-dependent manner (Mattner J. et al., Nature. 2005 Mar. 24; 434(7032):525-9; Kinjo Y. et al., Nature. 2005 Mar. 24; 434(7032):520-5). The biological response to these glycolipid antigens includes the production of IFNγ and IL-4 by type-I NKT cells.

Indirect Recognition and Activation of Type-I NKT Cells

Even though no cognate glycolipid antigens that are recognized by type-I NKT cell TCRs have been found in the main Gram-negative and Gram-positive bacterial pathogens that are prominent in human disease, alternative modes of type-I NKT cell activation have been reported for such bacteria. For example, LPS-positive bacteria like Salmonella or Escherichia have been shown to activate type-I NKT cells indirectly. These indirect means of recognition fall into two main groups: those that depend, at least partially, upon CD1d/TCR interactions in conjunction with the activation of antigen presenting cells, and those that appear to be CD1d-independent.

First, it was shown that Gram-negative bacteria (such as Salmonella typhimurium) or Gram-positive bacteria (such as Staphylococcus aureus) cultured with dendritic cells can stimulate type-I NKT cells in absence of specific cognate foreign glycolipids (Mattner J. et al., Nature. 2005 Mar. 24; 434(7032):525-9; Brigl M et al., Nat Immunol. 2003 December; 4(12):1230-7). Such stimulation is blocked by either anti-CD1d or anti-IL-12 mAbs in vitro and in vivo. These results suggest that a vast array of microorganisms might be able to induce type-I NKT activation indirectly through APC stimulation. This mechanism is dependent on TLR engagement of the APC as S. typhimurium-exposed wild-type derived bone marrow-derived dendritic cells (DCs), but not TLR-signaling molecules-deficient DCs, were able to stimulate type-I NKT cells in vitro (Mattner J. et al., Nature. 2005 Mar. 24; 434 (7032): 525-9). It is also likely dependent upon recognition of a self-glycolipid by the type-I NKT TCR because CD1-deficient DCs are unable to stimulate type-I NKT cells when stimulated similarly. Furthermore, APC activation by TLR ligands was shown to modulate the lipid biosynthetic pathway and to induce the specific upregulation of CD1d-bound ligand(s), as demonstrated using multimeric type-I NKT TCRs as a staining reagent (Salio M. et al., Proc Natl Acad Sci USA. 2007; 104: 20490-20495). In contrast with these results, it was reported that Escherichia coli LPS induces the stimulation of type-I NKT cells in an APC-dependent but CD1d-independent manner (Nagarajan N A. et al., J Immunol. 2007; 178:2706-2716). In these experiments, IFNγ-production by type-I NKT cells did not require the CD1d-mediated presentation of an endogeneous antigen, and exposure to a combination of IL-12 and IL-18 was sufficient to activate them.

Finally, it was reported that in addition to the LPS-detecting sensor TLR4, activation of the nucleic acid sensors TLR7 and TLR9 in DCs also leads to the stimulation of type-I NKT cells, as measured by their production of IFNγ (Paget C. et al., Immunity. 2007; 27:597-609).

### Type-I NKT Cells in Disease

Although type-I NKT cells represent a relatively low frequency of peripheral blood T cells in humans, their limited TCR diversity means that they respond at high frequency following activation. As such, type-I NKT cells are uniquely positioned to shape adaptive immune responses and have been demonstrated to play a modulatory role in a wide variety of diseases such as cancer, autoimmunity, inflammatory disorders, tissue transplant-related disorders, and infection (Terabe & Berzofsky, Ch. 8, Adv Cancer Res, 101: 277-348, 2008; Wu & van Kaer, Curr Mol Med, 9: 4-14, 2009; Tessmer et al, Expert Opin Ther Targets, 13: 153-162, 2009). For example, mice deficient in NKT cells are susceptible to the development of chemically induced tumors, whereas wild-type mice are protected (Guerra et al, Immunity 28: 571-80, 2008). These experimental findings correlate with clinical data showing that patients with advanced cancer have decreased type-I NKT cell numbers in peripheral blood (Gilfillan et al, J Exp Med, 205: 2965-73, 2008).

Type-I NKT cells constitute <0.1% of peripheral blood and <1% of bone marrow T cells in humans, but despite their relative scarcity, they exert potent immune regulation via production of IL-2, Th1-type (IFN-γ, TNF-α), Th2-type (IL-4, IL-13), IL-10, and IL-17 cytokines. (Lee et al, J Exp Med, 2002; 195: 637-641; Bendelac et al, Annu Rev Immunol, 2007; 178: 58-66; Burrows et al, Nat Immunol, 2009; 10(7): 669-71). Type-I NKT cells are characterized by a highly restricted (invariant) T-cell receptor (TCR)-Vα chain (Vα24 in humans). Their TCR is unique in that it recognizes altered glycolipids of cell membranes presented in context of a ubiquitous HLA-like molecule, CD1d. (Zajonc & Kronenberg, Immunol Rev, 2009; 230 (1): 188-200). CD1d is expressed at high levels on many epithelial and hematopoietic tissues and on numerous tumor targets, and is known to specifically bind only the type-I NKT TCR. (Borg et al, Nature, 2007, 448: 44-49).

Like NK cells, type-I NKT cells play a major role in tumor immunosurveillance, via direct cytotoxicity mediated through perforin/Granzyme B, Fas/FasL, and TRAIL pathways. (Brutkiewicz & Sriram, Crit Rev Oncol Hematol, 2002; 41: 287-298; Smyth et al, J. Exp. Med. 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237). In mice, type-I NKT cells protect against GVHD, while enhancing cytotoxicity of many cell populations including NK cells. Unlike NK cells, type-I NKT cells are not known to be inhibited by ligands such as Class I MHC, making them useful adjuncts in settings of tumor escape from NK cytotoxicity via Class I upregulation. (Brutkiewicz & Sriram, Crit Rev Oncol Hematol, 2002; 41: 287-298; Smyth et al, J Exp Med 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237).

Further evidence supporting a role for type-I NKT cells in antitumor immunity is provided in studies using Jα18 gene-targeted knockout mice that exclusively lack type-I NKT cells (Smyth et al, J Exp Med, 191: 661-668, 2000). For example, type-I NKT-deficient mice exhibited significantly increased susceptibility to methylcholanthrene-induced sarcomas and melanoma tumors, an effect reversed by the administration of liver-derived type-I NKT cells during the early stages of tumor growth (Crowe et al, J Exp Med, 196: 119-127, 2002).

At least one contribution of type-I NKT cells to antitumor immunity occurs indirectly via the activation of type-I NKT cells by DCs. Activated type-I NKT cells can initiate a series of cytokine cascades—including production of interferon gamma (IFN-γ)—that helps boost the priming phase of the antitumor immune response (Terabe &. Berzofsky, Ch 8, Adv Cancer Res, 101: 277-348, 2008). IFN-γ production by type-I NKT cells, as well as NK cells and CD8+ effectors, has been shown to be important in tumor rejection (Smyth et al, Blood, 99: 1259-1266, 2002). The underlying mechanisms are well characterized (Uemura et al, J Imm, 183: 201-208, 2009).

Further, type-I NKT cells have been shown to specifically target the killing of CD1d-positive tumor-associated macrophages (TAMs), a highly plastic subset of inflammatory cells derived from circulating monocytes that perform immunosuppressive functions (Sica & Bronte, J Clin Invest, 117: 1155-1166, 2007). TAMS are known to be a major producer of interleukin-6 (IL-6) that promotes proliferation of many solid tumors, including neuroblastomas and breast and prostate carcinomas (Song et al., J Clin Invest, 119: 1524-1536, 2009; Hong et al, Cancer, 110: 1911-1928, 2007). Direct CD1d-dependent cytotoxic activity of type-I NKT cells against TAMS suggests that important alternative indirect pathways exist by which type-I NKT cells can mediate antitumor immunity, especially against solid tumors that do not express CD1d.

In humans, type-I NKT cells home to neuroblastoma cells (Metelitsa et al, J Exp Med 2004; 199 (9):1213-1221) and B cell targets (Wilson & Delovitch, Nat Rev Immunol 2003; 3: 211-222; Molling et al, Clinical Immunology, 2008; 129: 182-194) both of which express high levels of CD1d. Type-I NKT cell cytokines may increase NK cytotoxicity. IFN-γ enhances NK cell proliferation and direct cytotoxicity, whereas IL-10 potently increases TIA-1, a molecule within NK cytotoxic granules which has direct DNA cleavage effects (Tian et al, Cell, 1991; 67 (3): 629-39) and can regulate mRNA splicing in NK cell targets, favoring expression of membrane-bound Fas on targets. (Izquierdo et al, Mol Cell, 2005; 19 (4): 475-84). IL-10 further enhances tumor target susceptibility to NK lysis by inducing tumor downregulation of Class I MHC, a major inhibitory ligand for NK cells. (Kundu & Fulton, Cell Immunol, 1997; 180:55-61).

Evidence supporting an important role for type-I NKT cells in the treatment of inflammatory diseases and/or autoimmune diseases comes from studies using murine autoimmune disease models. For example, in mouse models of type I diabetes (M. Falcone et al, J Immunol, 172: 5908-5916, 2004; Mizuno et al, J Autoimmun, 23: 293-300, 2004), rheumatoid arthritis (Kaieda et al, Arthritis and Rheumatism, 56: 1836-1845, 2007; Miellot-Gafsou et al, Immunology, 130: 296-306, 2010), autoimmune colitis (Crohn's disease and ulcerative colitis models DSS-induced colitis and autoimmune T cell-mediated colitis; Geremia et al., Autoimmun Rev. 13(1):3-10, 2014 doi: 10.1016/j.autrev.2013.06.004. Epub 2013 Jun. 15. Katsurada et al., PLoS One, 7(9):e44113, 2012; Fuss and Strober, Mucosal Immunol., 1 Suppl 1:S31-3, 2008), and experimental autoimmune encephalitis (EAE) (van de Keere & Tonegawa, J Exp Med, 188: 1875-1882, 1998; Singh et al, J Exp Med, 194:1801-1811, 2001; Miyamoto et al, Nature, 413: 531-534, 2001), type-I NKT cells played key roles in establishing immune tolerance and preventing autoimmune pathology.

Type-I NKT cells are also activated and participate in responses to transplanted tissue. Without subscribing exclusively to any one theory, evidence supports an important role for type-I NKT cells in transplantation-related disorders. For example, type-I NKT cells have been shown to infiltrate both cardiac and skin allografts prior to rejection and have been found in expanded numbers in peripheral lymphoid tissue following transplantation (Maier et al, Nat Med, 7: 557-62, 2001; Oh et al, J Immunol, 174: 2030-6, 2005; Jiang et al, J Immunol, 175: 2051-5, 2005). Type-I NKT cells are not only activated, but also influence the ensuing immune response (Jukes et al, Transplantation, 84: 679-81, 2007). For example, it has been found consistently that animals deficient in either total NKT cells or type-I NKT cells are resistant to the induction of tolerance by co-stimulatory/co-receptor molecule blockade (Seino et al, Proc Natl Acad Sci USA, 98: 2577-81, 2001; Jiang et al, J Immunol, 175: 2051-5, 2005; Jiang et al, Am J Transplant, 7: 1482-90, 2007). Notably, the adoptive transfer of NKT cells into such mice restores tolerance, which is dependent on interferon (IFN)-γ, IL-10 and/or CXCL16 (Seino et al, Proc Natl Acad Sci USA, 98: 2577-81, 2001; Oh et al, J Immunol, 174: 2030-6, 2005; Jiang et al, J Immunol, 175: 2051-5, 2005; Jiang et al, Am J Transplant, 7: 1482-90, 2007; Ikehara et al, J Clin Invest, 105: 1761-7, 2000). In addition, type-I NKT cells have proved to be essential for the induction of tolerance to corneal allografts and have been demonstrated to prevent graft-versus-host disease in an IL-4-dependent manner (Sonoda et al, J Immunol, 168: 2028-34, 2002; Zeng et al, J Exp Med, 189: 1073-81 1999; Pillai et al, Blood. 2009; 113:4458-4467; Leveson-Gower et al, Blood, 117: 3220-9, 2011).

Type-I NKT cell responses may depend on the type of transplant carried out, for example, following either vascularized (heart) or non-vascularized (skin) grafts, as the alloantigen drains to type-I NKT cells residing in the spleen or axillary lymph nodes, respectively. Further, type-I NKT cell responses can be manipulated, for example, by manipulating type-I NKT cells to release IL-10 through multiple injections of α-GalCer, which can prolong skin graft survival (Oh et al, J Immunol, 174: 2030-6, 2005).

Achievement of allogeneic immune tolerance while maintaining graft-versus-tumor (GVT) activity has previously remained an elusive goal of allogeneic hematopoietic cell transplantation (HCT) Immune regulatory cell populations including NKT cells and CD4⁺Foxp3⁺ regulatory T (Treg) cells are thought to play a key role in determining tolerance and GVT. To this end, reduced intensity conditioning methods which enrich for NKT and Treg cells have recently been applied with some measure of success. Specifically, a regimen of total lymphoid irradiation (TLI) and anti-thymocyte globulin (ATG) has resulted in engraftment and protection from graft-versus-host disease (GVHD) in both children and adults (Lowsky et al, New England Journal of Medicine. 2005, 353:1321-1331; Kohrt et al, Blood. 2009; 114:1099-1109; Kohrt et al, European Journal of Immunology. 2010; 40:1862-1869; Pillai et al, Pediatric Transplantation. 2011; 15:628-634) and GVT appeared to be maintained in adult patients whose disease features rendered them at high risk for relapse (Lowsky et al, The New England Journal of Medicine. 2005, 353:1321-1331; Kohrt et al, Blood. 2009; 114:1099-1109; Kohrt et al, European Journal of Immunology. 2010; 40:1862-1869).

Murine pre-clinical modeling of this regimen showed that GVHD protection is dependent upon the IL-4 secretion and regulatory capacity of type-I NKT cells, and that these cells regulate GVHD while maintaining GVT (Pillai et al, Journal of Immunology. 2007; 178:6242-6251). Further, type-I NKT derived IL-4 results can drive the potent in vivo expansion of regulatory CD4⁺CD25⁺Foxp3⁺ Treg cells, which themselves regulate effector CD8⁺ T cells within the donor to prevent lethal acute GVHD (Pillai et al, Blood. 2009; 113:4458-4467). It has been shown that type-I NKT cell-dependent immune deviation results in the development and augmentation of function of regulatory myeloid dendritic cells, which in turn induce the potent in vivo expansion of regulatory CD4+CD25+Foxp3+ Treg cells and further enhance protection from deleterious T cell responses (van der Merwe et al, J. Immunol., 2013; Nov. 4, 2013).

In response to infection, the immune system relies upon a complex network of signals through the activation of receptors for pathogen-associated molecular patterns, such as the Toll-like receptors (TLRs), expressed on antigen-presenting cells (APC), consequently promoting antigen-specific T cell responses (Medzhitov & Janeway Jr, Science 296: 298-300, 2002). For example, during such responses, type-I NKT cells respond through the recognition of microbial-derived lipid antigens, or through APC-derived cytokines following TLR ligation, in combination with, and without the presentation of, self- or microbial-derived lipids. Bacterial antigens can also directly stimulate type-I NKT cells when bound to CD1d, acting independently of TLR-mediated activation of APC (Kinjo et al, Nat Immunol, 7: 978-86, 2006; Kinjo et al, Nature, 434:520-5, 2005; Mattner et al, Nature, 434: 525-9, 2005; Wang et al, Proc Natl Acad Sci USA, 107: 1535-40, 2010).

Further, NKT (CD1d−/−) and type-I NKT (Jα18−/−) cell-deficient mice have been shown to be highly susceptible to influenza compared with wild-type mice (De Santo et al, J Clin Invest, 118: 4036-48, 2008). In this model, type-I NKT cells were found to suppress the expansion of myeloid-derived suppressor cells (MDSC) which were expanded in CD1d and Jα18−/− mice (Id.). Importantly, although the exact mechanism of type-I NKT cell activation was not determined, the authors suggest that type-I NKT cells required TCR-CD1d interactions, as the adoptive transfer of type-I NKT cells to Jα18−/− but not CD1d−/− mice suppressed MDSC expansion following infection with PR8 (De Santo et al, J Clin Invest, 118:4036-48, 2008). Thus another application of type-I NKT cells is in augmentation of immune responses to pathogens (e.g., bacterial, viral, protozoal, and helminth pathogens).

Finally, type-I NKT cells have been shown to play a critical role in regulating and/or augmenting the allergic immune response, both through secretion of cytokines and through modulation of other immune subsets including regulatory Foxp3+ cells, APCs, and NK cells (Robinson, J Allergy Clin Immunol., 126(6):1081-91, 2010; Carvalho et al., Parasite Immunol., 28(10):525-34, 2006; Koh et al., Hum Immunol., 71(2):186-91, 2010). This includes evidence in atopic dermatitis models (Simon et al., Allergy, 64(11):1681-4, 2009).

However, a major obstacle to application of human innate regulatory type-I NKT cells in immunotherapy is their relative scarcity in common cellular therapy cell products including human peripheral blood (Berzins et al, Nature Reviews Immunology. 2011; 11:131-142; Exley et al, Current Protocols in Immunology, 2010; Chapter 14: Unit 14-11; Exley & Nakayama, Clinical Immunology, 2011; 140:117-118) and the lack of clear phenotypic and functional data on ex vivo expanded human type-I NKT cells to validate the potential application of post-expansion human type-I NKT cells therapeutically.

Despite the great immunological importance and therapeutic potential of type-I NKT cells, the art lacks technologies necessary to efficiently expand and/or modulate the activity of type-I NKT cells ex vivo sufficient to allow their use in therapeutic methods.

### SUMMARY OF THE INVENTION

According to one aspect the described invention provides to pharmaceutical composition comprising a pharmaceutically acceptable carrier and a cell product comprising an expanded and enriched population of superactivated cytokine killer cells (SCKTCs) derived from a population of cytokine killer T cells, the SCKTCs characterized by two or more of an induced secretion of a cytokine, a stimulated proliferation of the population of SCKTCs, an improved cytotoxicity of the SCKTCs, and modulated expression of one or more markers on the cell surface of the SCKTCs, compared to an unstimulated, unactivated cytokine killer T cell control population. According to one embodiment, the cytokine whose expression is modulated is one or more selected from the group consisting of IL-4, IL-5, IL-6, or IL-10 and IFNγ. According to another embodiment, the expanded and enriched population of SCKTCs comprises low expression of one or more cytokines selected from the group consisting of IL-4, IL-5, IL-6, and IL-10, and high expression of IFNγ. According to another embodiment, cytokine production by the expanded and enriched population of SCKTCs is characterized as Il-5-, IL-6-, Il-10-, IL-4 low, IFNγ high. According to another embodiment, the amount of IFN-γ produced by the expanded and enriched population of SCKTCs is about 5000 pg/ml or greater. According to another embodiment, the amount of IL-4 produced by the expanded and enriched population of SCKTCs is less than 5 pg/ml. According to another embodiment, a ratio of IFNγ: IL-4 in culture supernatants of the expanded and enriched population of SCKTCs is equal to or greater than 1000. According to another embodiment, a killing rate of a target cell by the expanded and enriched population of SCKTCs ranges from about 25% to about 75%, inclusive. According to another embodiment, the killing rate of the expanded and enriched population of SCKTCs is at least 1.5 fold greater than the killing rate of nonexpanded, nonactivated cytokine killer T cell control cells. According to another embodiment, a ratio of IFN-γ: IL-4 is at least 1000, and the killing rate is increased at least 1.5 fold greater by the expanded and enriched population of SCKTCs compared to the killing rate of nonexpanded, nonactivated cytokine killer T cell control cells. According to another embodiment, the expanded and enriched population of SCKTCs comprises a subpopulation of SCKTCs that express NKT cell markers. According to another embodiment, the expanded and enriched population of SCKTCs cells comprises a subpopulation of SCKTCs comprising one or more of CD3+Vα24+ cells, CD3+Vα24− cells or CD3+CD56+ cells. According to another embodiment, the expanded and enriched population of SCKTCs comprises a subpopulation of SCKTCs that are CD3+CD56+. According to another embodiment, the expanded and enriched population of SCKTCs comprises a subpopulation of SCKTCs that express type 1 NKT cells markers. According to another embodiment, the type 1-NKT cell markers comprise TCR Vα and TCR Vβ markers. According to another embodiment, the subpopulation of SCKTCs that express type 1 NKT cells markers comprises cells characterized as CD3+Vα24+, CD3+Vα24-, or CD3+CD56+. According to another embodiment, the expanded and enriched population of SCKTCs derived from a population of cytokine killer T cells (CKTCs) constitutes from about 40% to about 60% of the total CKTC population. According to another embodiment, the pharmaceutical composition comprises a stabilizing amount of serum that is effective for retention by the expanded and enriched population of SCKTCs of their T cell effector activity. According to another embodiment, the stabilizing amount of serum is at least 10%. According to another embodiment, the serum is human serum.

According to another aspect, the described invention provides a method for preparing a pharmaceutical composition comprising an expanded and enriched population of superactivated cytokine killer T cells (SCKTCs) comprising, in order

(a) isolating a population of mononuclear cells (MCs) comprising a population of cytokine killer T cells (CKTCs);

(b) optionally transporting the preparation of (a) to a processing facility under sterile conditions;

(c) culturing the population of MCs in a culture system;

(d) contacting the culture system of step (c) with alpha-galactosylceramide (αGalCer), or an analog or functional equivalent thereof, and with a population of cells comprising CD1d and αGalCer or an analog or functional equivalent thereof, wherein the contacting is sufficient to stimulate expansion of the population of CKTCs;

(e) contacting the culture system of step (d) with IL-2, IL-7, IL-15 and IL-12, in a predetermined order and time of addition, together with pulses of a fresh population of cells comprising CD1d and αGalCer, wherein the contacting is sufficient to stimulate activation of some of the population of CTKCs and to form the expanded and enriched population of SCKTCs;

(f) collecting the expanded and enriched population of SCKTCs from the culture system to form an SCKTC cell product; wherein the cell product comprising the expanded and enriched population of SCKTCs of (f) is characterized by one or more of an improved ability to secrete effector cytokines or improved cytotoxicity compared to the population of CKTCs of (a); and

(h) formulating the cell product with a pharmaceutically acceptable carrier to form the pharmaceutical composition.

According to one embodiment, a source of the mononuclear cells (MCs) in (a) is blood. According to another embodiment, the MCs are derived from a human subject. According to another embodiment, the MCs are isolated from whole blood by Ficoll-Paque gradient centrifugation. According to another embodiment, the method comprises between steps (e) and (f) transporting the culture from the processing facility to a treatment facility. According to another embodiment, the transporting step is initiated within from about 1 hour to about 24 hours after addition of IL12. According to another embodiment, step (c) optionally comprises re-suspending the MCs and adjusting the MCs to a concentration ranging from about 5×10⁵ cells/ml to about 3×10⁶ cells/ml before performing step (d). According to another embodiment, step (e) comprising adding pulses of a fresh population of cells comprising CD1d and αGalCer or an analog or functional equivalent thereof to the culture system. According to some embodiments, the number of pulses of the fresh population of cells comprising CD and αGalCer is at least 1, at least 2, at least 3, at least 4, at least 5, at least 6, at least 7, at least 8, at least 9, or at least 10. According to another embodiment, the αGalCer, or an analog or functional equivalent thereof is maintained at a constant concentration from step (d) to step (f). According to another embodiment, the concentration of αGalCer, or an analog or functional equivalent thereof, is between about 50 ng/ml to about 500 ng/ml. According to another embodiment, IL-2 is maintained at a constant concentration from step (e) to step (f). According to another embodiment, the concentration of IL-2 ranges from about 10 U/ml to about 100 U/ml. According to another embodiment, the IL-7 is maintained at a constant concentration from step (e) to step (f). According to another embodiment, the concentration of IL-7 ranges from about 20 ng/ml to 200 ng/ml. According to another embodiment, IL-2 and IL-7 are added at about day 7 of culture. According to another embodiment, IL-15 is added at about day 14 of culture. According to another embodiment, the IL-12 is added at about day 20 of culture. According to another embodiment, step (f) is carried out at least about day 21 of culture. According to another embodiment, the IL-15 is maintained at a constant concentration from step (e) to step (f). According to another embodiment, the concentration of IL-15 ranges from about 10 ng/ml to about 100 ng/ml. According to another embodiment, the IL-12 is maintained at a constant concentration from step (e) to step (f). According to another embodiment, the concentration of IL-12 ranges from about 10 ng/ml to about 100 ng/ml. According to another embodiment, the method further comprises a step of characterizing expression of cell surface markers by the population of SCKTCs by flow cytometry. According to another embodiment, a subpopulation of the expanded and enriched population of SCKTCs comprises one or more of CD3+Vα24+ cells, CD3+Vα24− cells or CD3+CD56+ cells. According to another embodiment, the subpopulation further comprises Vβ11+ cells. According to one embodiment, the expanded and enriched population of SCKTCs comprises a subpopulation of CD3+Vα24+Vβ11+ cells, CD3+Vα24− cells, or CD3+CD56+ cells.

According to another embodiment, the expanded and enriched population of SCKTCs comprises from about 40% to about 60% of the total population of CKTCs. According to another embodiment, IL-2 and IL-7 are added to the culture simultaneously. According to another embodiment, IL-2, IL-7 and IL-15 are added to the culture simultaneously. According to another embodiment, the population of MCs in step (c) comprises from about 5×10⁵ cells/ml to about 3×10⁶ cells/ml. According to another embodiment, the cell comprising CD1d and alpha-galactosylceramide (αGalCer) is an antigen presenting cell. According to another embodiment, the antigen presenting cell is a dendritic cell (DC). According to another embodiment, the dendritic cell is loaded with αGalCer. According to another embodiment, the dendritic cell loaded with αGalCer is derived from the MCs and is an adherent cell. According to another embodiment, the dendritic cell loaded with αGalCer is prepared by a method comprising: (a) isolating a population of mononuclear cells (MCs); (b) culturing the population of MCs in a culture system; (c) contacting the culture system with IL-4 and GM-CSF, wherein the contacting is sufficient to induce differentiation of the MCs into dendritic cells; and (d) contacting the culture system with αGalCer, wherein the contacting is sufficient to load the dendritic cells with αGalCer. According to another embodiment in the method for preparing the dendritic cell loaded with αGalCer, the dendritic cell loaded with αGalCer is an adherent cell. According to another embodiment, in the method for preparing the dendritic cell loaded with αGalCer, the concentration of IL-4 is 500 U/ml. According to another embodiment, in the method for preparing the dendritic cell loaded with αGalCer, the concentration of GM-CSF is 50 ng/ml. According to another embodiment, in the method for preparing the dendritic cell loaded with αGalCer, step (d) is carried out from about 5 days to about 7 days after step (b). According to another embodiment, in the method for preparing the dendritic cell loaded with αGalCer, the population of MCs in step (b) comprise from about 1×10⁵ cells/ml to about 5×10⁶ cells/ml. According to another embodiment in the method for preparing the dendritic cell loaded with αGalCer, steps (b)-(d) are carried out in a culture medium selected from RPMI 1640 medium containing 10% fetal bovine serum or 10% autologous serum.

According to another embodiment the method for preparing the composition further comprises replenishing the culture medium in the culture system every 2 to 3 days. According to another embodiment, steps (c)-(f) are carried out in a culture medium selected from X-VIVO-15 serum-free medium, RPMI 1640 medium containing 10% fetal bovine serum or 10% autologous serum.

According to another aspect the described invention provides a pharmaceutical composition comprising a pharmaceutically acceptable carrier and a cell product comprising an enhanced and enriched population of superactivated cytokine killer T cells (SCKTCs) produced by the described and claimed method. According to one embodiment of the pharmaceutical composition produced by the method described herein, the expanded and enriched population of SCKTCs comprises a subpopulation of CD3+Vα24+Vβ11+ cells, CD3+Vα24− cells, or CD3+CD56+ cells. According to another embodiment, the subpopulation further comprises Vβ11+ cells. According to one embodiment, the expanded and enriched population of SCKTCs comprises a subpopulation of CD3+Vα24+Vβ11+ cells, CD3+Vα24− cells, or CD3+CD56+ cells.

According to some embodiments, the pharmaceutical composition further comprises an additional therapeutic agent selected from the group consisting of a chemotherapeutic agent, a biological response modifying agent, and an immunotherapeutic agent.

According to some embodiment, the immunotherapeutic agent is an antibody. According to some embodiments, the antibody is a monoclonal antibody, a humanized antibody, a human antibody or a chimeric antibody.

The compositions and methods described by the present disclosure provide a number of advantages over current immunotherapies. For example, while CAR-T therapy holds promise for the treatment of various cancers, CAR-T therapy comes with a number of disadvantages. CAR T-cell therapy can trigger a range of side effects, many of which begin subtly, but can rapidly worsen. A particularly severe complication is cytokine release syndrome (CRS), also known as a cytokine storm. Once CAR-T cells enter the body, they initiate a massive release of cytokines, which summon other elements of the immune system to join the attack on tumor cells. CRS is characterized by fever, hypotension and respiratory insufficiency associated with elevated serum cytokines, including interleukin-6 (IL-6) (Davila et al., Sci. Transl. Med. 6, 224ra25 (2014); CRS usually occurs within days of T cell infusion at the peak of CAR T cell expansion. The condition tends to be especially severe in patient with extensive cancers.

The compositions and methods of the present invention advantageously bypass the problem of CRS, because the infused cell product is self, and the cytokine storm has been consigned to cell culture.

### BRIEF DESCRIPTION OF THE DRAWINGS

FIGS. 1A and 1B show the results of flow cytometry experiments to determine the proportion of SCKTC target cells in the expanded population of CTKCs in Example 3; FIG. 1A shows the proportion of cells expressing markers of CD3+CD56+ cells. FIG. 1B shows the proportion of cells expressing markers of type-I NKT cells.

FIGS. 2A-D show the effect of time of adding cytokines IL-12 and IL-7 on the proportion of cells expressing markers of type-I NKT cells in the expanded population of CTKCs in Example 4. Flow cytometry was used to determine the presence of cells expressing the markers TCR Vα24 (Vα24) and TCR Vβ11 (Vb11), where a gate was set based on Vα24+Vb11+ cells. FIG. 2A shows the results for Group A, where IL-2 was added at the beginning of culture. FIG. 2B shows the results for Group B, where IL-2 and IL-7 were added simultaneously at the beginning of culture. FIG. 2C shows the results for Group C, where IL-2 and IL-7 were added at day 3 of culture. FIG. 2D shows the results for Group D, where IL-2 and IL-7 were added at day 7 of culture.

FIGS. 3A-D show the effect of time of adding cytokine IL-15 on the proportion of cells expressing markers of type-I NKT cells in the expanded population of CTKCs in Example 5. Flow cytometry was used to determine the presence of cells expressing TCR Vα24 (Vα24) and TCR Vβ11 (Vb11), where a gate was set based on Vα24+Vb11+ cells. FIG. 3A shows the results for Group A, where IL-2 and IL-7 were added simultaneously at day 7 of culture and IL-15 was not added. FIG. 3B shows the results for Group B, where IL-2 and IL-7 were added simultaneously at day 7 of culture and IL-15 was added at the beginning of culture. FIG. 3C shows the results for Group C, where IL-2 and IL-7 were added simultaneously at day 7 of culture and IL-15 was added at day 7 of culture. FIG. 3D shows the results for Group D, where IL-2 and IL-7 were added simultaneously at day 7 of culture and IL-15 was added at day 14 of culture.

FIGS. 4A-D show the effect of time of adding cytokine IL-12 on the proportion of cells expressing markers of type-I NKT cells in the expanded population of CTKCs in Example 5. Flow cytometry was used to determine the presence of cells expressing TCR Vα24 (Vα24) and TCR Vβ11 (Vb11), where a gate was set based on Vα24+Vb11+ cells. FIG. 4A shows the results for Group A, where IL-2 and IL-7 were added simultaneously at day 7 of culture, IL-15 was added at day 14 of culture, and no IL-12 was added. FIG. 4B shows the results for Group B, where IL-2 and IL-7 were added simultaneously at day 7 of culture, IL-15 was added at day 14 of culture, and IL-12 was added at the beginning of culture. FIG. 4C shows the results for Group C, where IL-2 and IL-7 were added simultaneously at day 7 of culture, IL-15 was added at day 14 of culture, and IL-12 was added at day 7 of culture. FIG. 4D shows the results for Group D, where IL-2 and IL-7 were added simultaneously at day 7 of culture, IL-15 was added at day 14 of culture, and IL-12 was added at day 20 of culture.

### DETAILED DESCRIPTION

The present disclosure is based, in part, on the discovery of an ex vivo method for preparing a pharmaceutical composition comprising a cell product comprising an expanded and enriched population of superactivated cytokine killer T cells (SCKTCs) with an improved ability to secrete effector cytokines and improved cytotoxicity. Thus, the present disclosure provides in vitro methods for generation of large numbers of functional SCKTCs, which can be further used for adoptive transfers.

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

### Definitions

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer. According to one embodiment, to A without B (optionally including elements other than B). In some embodiments, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

As used herein, “substantially equal” means within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

As used herein, the terms “activating or activate cytokine killer T cells” or “CKTCl activation” is meant to refer to a process causing or resulting in one or more cellular responses of CKTCs, including: proliferation, differentiation, cytokine secretion, cytotoxic effector molecule release, cytotoxic activity, and expression of activation markers. As used herein, an “activated cytokine killer T cell” refers to a cytokine killer T cell that has received an activating signal, and thus demonstrates one or more cellular responses, including proliferation, differentiation, cytokine secretion, cytotoxic effector molecule release, cytotoxic activity, and expression of activation markers. The activating of the CKTC can comprise one or more of inducing secretion of a cytokine from the CKTC, stimulating proliferation of the CKTC, and upregulating expression of a cell surface marker on the CKTC. The cytokine can be one or more of IL-1, IL-2, IL-4, IL-5, IL-6, IL-10, IL-13, IL-15, TNF-α, TNF-β, and IFN-γ. According to certain embodiments, activating of a CKTC can comprise secretion of one or more of, IL-4, IL-5, 11-6, IL-10, or IFN-γ. Suitable assays to measure CKTC activation are known in the art and are described herein.

The term “active” refers to the ingredient, component or constituent of the pharmaceutical compositions of the described invention responsible for an intended therapeutic effect.

As used herein, the term “administration” and its various grammatical forms as it applies to a mammal, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. “Administration” also encompasses in vitro and ex vivo treatments, e.g., of a cell, by a reagent, diagnostic, binding composition, or by another cell.

As used herein, the term “adaptive cellular therapy” or “adaptive transfer” refer to a treatment used to help the immune system fight diseases by which T cells collected from a patient are expanded (grown in a laboratory in culture) to increase the number of T cells able to fight the disease. These T cells then are given back to the patient.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human V_{L} chains with the heavy chain variable region (V_{H}) domain of a mouse monoclonal antibody reactive with an antigen of interest; (ii) selecting half-human Fabs on that antigen (iii) using the selected V_{L} genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1 antibody molecule in the mouse myeloma.

As used herein, the term “antigen presentation” refers to the display of antigen on the surface of a cell in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen. An APC can be an “artificial APC,” meant to refer to a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

As used herein, the term “autologous” is meant to refer to being derived from the same individual. As used herein the term “allogeneic” is meant to refer to being derived from two genetically different individuals.

As used herein, the term “autophagy” refers to the digestion and breakdown by a cell of its own organelles and proteins in lysosomes.

As used herein, the term “biomarker” (or “biosignature”) refers to a peptide, protein, nucleic acid, antibody, gene, metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

As used herein, the term “cancer” is meant to refer to diseases in which abnormal cells divide without control and are able to invade other tissues. There are more than 100 different types of cancer. Most cancers are named for the organ or type of cell in which they start—for example, cancer that begins in the colon is called colon cancer; cancer that begins in melanocytes of the skin is called melanoma. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system cancers (meaning cancers that begin in the tissues of the brain and spinal cord). The term “myelodysplastic syndrome” refers to a type of cancer in which the bone marrow does not make enough healthy blood cells (white blood cells, red blood cells, and platelets) and there are abnormal cells in the blood and/or bone marrow. Myelodysplastic syndrome may become acute myeloid leukemia (AML).

As used herein the term “CD1d” is meant to refer to a family of transmembrane glycoproteins, which are structurally related to the MHC proteins and form heterodimers with beta-2-microglobulins that mediate the presentation of primarily lipid and glycolipid antigens of self or microbial origin to T cells.

As used herein, the term “chemokine” is meant to refer to a class of chemotactic cytokines that signal leukocytes to move in a specific direction.

As used herein, the term “component” is meant to refer to a constituent part, element or ingredient.

As used herein, the term “composition” is meant to refer to a material formed by a mixture of two or more substances.

The term “condition”, as used herein, refers to a variety of health states and is meant to include disorders or diseases caused by any underlying mechanism or disorder.

As used herein, the term “contact” and its various grammatical forms is meant to refer to a state or condition of touching or of immediate or local proximity. Contacting a composition to a target destination may occur by any means of administration known to the skilled artisan.

As used herein, the term “costimulatory molecule” is meant to refer to one or two or more groups of atoms bonded together that are displayed on the cell surface of an APC that have a role in activating a naïve T cell to become an effector cell. For example MHC proteins, which present foreign antigen to the T cell receptor, also require costimulatory proteins which bind to complementary receptors on the T cell's surface to result in activation of the T cell.

As used herein the term “co-stimulatory receptor” is meant to refer to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

As used herein, the term “cognate help” is meant to refer to a process that occurs most efficiently in the context of an intimate interaction with a helper T cell.

As used herein, the term “culture” and its other grammatical forms is meant to refer to a process whereby a population of cells is grown and proliferated on a substrate in an artificial medium.

As used herein, the term “cytokine” refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Cytokines can act both locally and distantly from a site of release. They include type-I cytokines, which encompass many of the interleukins, as well as several hematopoietic growth factors; type-II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groα, MCP-1 and TNF-α.

As used herein, the term “dendritic cell” or “DC” describes a diverse population of morphologically similar cell types found in a variety of lymphoid and non-lymphoid tissues that present foreign antigens to T cells, see Steinman, Ann. Rev. Immunol. 9:271-296 (1991). As used herein, the term “derived from” is meant to encompass any method for receiving, obtaining, or modifying something from a source of origin.

As used herein, the term “detectable marker” is meant to refer to both selectable markers and assay markers. The term “selectable markers” is meant to refer to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

As used herein, the term “detectable response” is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The terms “disease” or “disorder” as used herein refer to an impairment of health or a condition of abnormal functioning.

As used herein, the term “dose” is meant to refer to the quantity of a therapeutic substance prescribed to be taken at one time. The term “maximum tolerated dose” as used herein is meant to refer to the highest dose of a drug or treatment that does not cause unacceptable side effects.

The term “endogenous” as used herein refers to any material from or produced inside an organism, cell, tissue or system.

As used herein, the term “enrich” is meant to refer to increasing the proportion of a desired substance, for example, to increase the relative frequency of a subtype of cell compared to its natural frequency in a cell population. Positive selection, negative selection, or both are generally considered necessary to any enrichment scheme. Selection methods include, without limitation, magnetic separation and FACS. Regardless of the specific technology used for enrichment, the specific markers used in the selection process are critical, since developmental stages and activation-specific responses can change a cell's antigenic profile.

As used herein, the terms “expanding a population of cytokine killer T cells (CKTCs)” or “cytokine killer T cell (CKTC)expansion” are meant to refer to a process wherein a population of cytokine killer T cells undergoes a series of cell divisions and thereby expands in cell number (for example, by in vitro culture). The term “expanded superactivated cytokine killer T cells” relates to superactivated cytokine killer T cells obtained through cell expansion.

As used herein, the term “expression” is meant to encompass production of an observable phenotype by a gene, usually b directing the synthesis of a protein. It includes the biosynthesis of mRNA, polypeptide biosynthesis, polypeptide activation, e.g., by post-translational modification, or an activation of expression by changing the subcellular location or by recruitment to chromatin.

As used herein the term “Fas” is meant to refer to a type 2 membrane protein found on lymphocytes that belongs to the TNF superfamily. In cells that express Fas, engagement of the cell death receptor Fas by Fas ligand (FasL) results in apoptotic cell death, mediated by caspase activation.

As used herein, the term “flow cytometry” is meant to refer to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether a cell is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

As used herein, the terms “formulation” and “composition” are used interchangeably herein to refer to a product of the present invention that comprises all active and inert ingredients. The terms “pharmaceutical formulation” or “pharmaceutical composition” as used herein refer to a formulation or composition that is employed to prevent, reduce in intensity, cure or otherwise treat a target condition or disease.

As used herein, the term “functional equivalent” or “functionally equivalent” are used interchangeably herein to refer to substances, molecules, polynucleotides, proteins, peptides, or polypeptides having similar or identical effects or use.

As used herein, the term “cell growth” is the process by which cells accumulate mass and increase in physical size. There are many different examples in nature of how cells can grow. In some cases, cell size is proportional to DNA content. For instance, continued DNA replication in the absence of cell division (called endoreplication) results in increased cell size. Megakaryoblasts, which mature into granular megakaryocytes, the platelet-producing cells of bone marrow, typically grow this way. By a different strategy, adipocytes can grow to approximately 85 to 120 μm by accumulating intracellular lipids. In contrast to endoreplication or lipid accumulation, some terminally differentiated cells, such as neurons and cardiac muscle cells, cease dividing and grow without increasing their DNA content. These cells proportionately increase their macromolecule content (largely protein) to a point necessary to perform their specialized functions. This involves coordination between extracellular cues from nutrients and growth factors and intracellular signaling networks responsible for controlling cellular energy availability and macromolecular synthesis. Perhaps the most tightly regulated cell growth occurs in dividing cells, where cell growth and cell division are clearly separable processes. Dividing cells generally must increase in size with each passage through the cell division cycle to ensure that a consistent average cell size is maintained. For a typical dividing mammalian cell, growth occurs in the G1 phase of the cell cycle and is tightly coordinated with S phase (DNA synthesis) and M phase (mitosis). The combined influence of growth factors, hormones, and nutrient availability provides the external cues for cells to grow. Guertin, D. A., Sabatini, D. M., “Cell Growth,” in The Molecular Basis of Cancer (4^{th} Edn) Mendelsohn, J. et al Eds, Saunders (2015), 179-190.

As used herein, the term “cell proliferation” is meant to refer to the process that results in an increase of the number of cells, and is defined by the balance between cell divisions and cell loss through cell death or differentiation.

As used herein, the term granulocyte-macrophage colony-stimulating factor” (GM-CSF) is meant to refer to a cytokine that promotes the proliferation and differentiation of hematopoietic progenitor cells and the generation of neutrophils, eosinophils, and macrophages. In synergy with other cytokines such as stem cell factor, IL-3, erythropoietin, and thrombopoietin, it also stimulates erythroid and megakaryocyte progenitor cells (Barreda, D R, et al, Developmental & Comparative Immunol. (2004) 28(50: 509-554). GM-CSF is produced by multiple cell types, including stromal cells, Paneth cells, macrophages, dendritic cells (DCs), endothelial cells, smooth muscle cells, fibroblasts, chondrocytes, and Th1 and Th17 T cells (Francisco-Cruz, A. et al, Medical Oncology (2014) 31: 774 et al.).

As used herein, the terms “immune response” and “immune-mediated” are used interchangeably herein and meant to refer to any functional expression of a subject's immune system, against either foreign or self-antigens, whether the consequences of these reactions are beneficial or harmful to the subject.

As used herein, the terms “immunomodulatory”, “immune modulator” and “immune modulatory” are used interchangeably herein to refer to a substance, agent, or cell that is capable of augmenting or diminishing immune responses directly or indirectly by expressing chemokines, cytokines and other mediators of immune responses.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

As used herein, the term “interferon gamma” (IFN-γ) is meant to refer to a soluble cytokine that is a member of the type II interferon class, which is secreted by cells of both the innate and adaptive immune systems. The active protein is a homodimer that binds to the interferon gamma receptor, which triggers a cellular response to viral and microbial infections.

As used herein, the term “interleukin-2” (IL-2) is meant to refer to a type of cytokine made by a type of T-lymphocyte that increases the growth and activity of other T lymphocytes and B lymphocytes and affects the development of the immune system. IL-2 made in the laboratory is called aldesleukin.

As used herein, the term “interleukin 4” (IL-4) is a pleiotropic cytokine whose actions are generally antagonistic to those of interferon gamma. Because IL-4R is widely expressed, IL-4 influences almost all cell types. In T cells, IL-4 is crucial for the differentiation and growth of the Th2 subset. As such, IL-4 promotes the establishment of the humoral response necessary to combat pathogens that live and reproduce extracellularly. In B cells, IL-4 stimulates growth and differentiation and induces upregulation of MHC class II and FcεRII (CD23). IL-4 also promotes isotype switching in murine B cells to IgG1 and IgE but inhibits switching to IgG2a, IgG2b, and IgG3. IL-4 is a growth factor for mast cells and plays a major regulatory role in allergic responses since these involve IgE-mediated mast cell degranulation. IL-4 is also important for defense against helminth worms because the IgE production promoted by IL-4 allows eosinophils bearing FcεRIIB to carry out efficient ADCC. In macrophages, IL-4 inhibits the secretion of pro-inflammatory chemokines and cytokines such as TNF and IL-1β, impairs the ability of these cells to produce reactive oxygen and nitrogen intermediates, and blocks IFNγ-induced expression of cellular adhesion molecules such as ICAM and E-selectin. However, IL-4 can also induce DCs and macrophages to upregulate their synthesis of IL-12, supplying a negative feedback mechanism to regulate the Th2 response. Mak, T W, Saunders, M E, Chapter 17, “Cytokines and Cytokine Receptors,” in The Immune Response, Basic and Clinical Principles (2006), Academic Press, pp. 463-516).

As used herein, the terms “interleukin-7” (IL-7) or lymphopoietin-1) are meant to refer to a type of cytokine made by cells that cover and support organs, glands and other structures in the body that causes the growth of T lymphocytes and B lymphocytes.

As used herein, the term “interleukin-12” (IL-12) is meant to refer to a type of cytokine made mainly by B lymphocytes and macrophages that causes other immune cells to make cytokines and increase the growth of T lymphocytes. It may also block the growth of new blood vessels.

As used herein, the term “interleukin-15” (IL-15) is meant to refer to a type of cytokine that acts through its specific receptor, IL-15Rα, which is expressed on antigen-presenting dendritic cells, monocytes and macrophages. IL-15 regulates T and natural killer cell activation and proliferation. IL-15 and IL-2 share many biological activities. They are found to bind common hematopoietin receptor subunits, and may compete for the same receptor, and thus negatively regulate each other's activity. The number of CD8+ memory cells is shown to be controlled by a balance between IL-15 and IL2. IL-15 induces the activation of JAK kinases, as well as the phosphorylation and activation of transcription activators STAT3, STAT5, and STAT6. Studies of the mouse counterpart suggested that IL-15 may increase the expression of apoptosis inhibitor BCL2L1/BCL-x(L), possibly through the transcription activation activity of STAT6, and thus prevent apoptosis.

As used herein, the term “isolated” is meant to refer to the separation of cells from a population through one or more isolation methods such as, but not limited to, mechanical separation or selective culturing. An “isolated” population of cells does not have to be pure. Other cell types may be present. According to some embodiments, and isolated population of a particular cell type refers to greater than 10% pure, greater than 20% pure, greater than 30% pure, greater than 40% pure, greater than 50% pure, greater than 60% pure, greater than 70% pure, greater than 80% pure, greater than 90% pure, or greater than 95% pure.

As used herein, the term “Kaplan Meier plot” or “Kaplan Meier survival curve” is meant to refer to the plot of probability of clinical study subjects surviving in a given length of time while considering time in many small intervals. The Kaplan Meier plot assumes that: (i) at any time subjects who are censored (i.e., lost) have the same survival prospects as subjects who continue to be followed; (ii) the survival probabilities are the same for subjects recruited early and late in the study; and (iii) the event (e.g., death) happens at the time specified. Probabilities of occurrence of events are computed at a certain point of time with successive probabilities multiplied by any earlier computed probabilities to get a final estimate. The survival probability at any particular time is calculated as the number of subjects surviving divided by the number of subjects at risk. Subjects who have died, dropped out, or have been censored from the study are not counted as at risk.

As used herein, the term “labeling” is meant to refer to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

As used herein, the terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

As used herein, the term “MHC (major histocompatibility complex) molecule” refers to one of a large family of ubiquitous cell-surface glycoproteins encoded by genes of the major histocompatibility complex (MHC). They bind peptide fragments of foreign antigens and present them to T cells to induce an immune response.” Class I MHC molecules, which are encoded by a series of highly polymorphic genes, are present on almost all cell types and present viral peptides on the surface of virus-infected cells, where they are recognized by cytotoxic T cells. In the MHC class I mechanism, foreign peptides are endocytosed for transport within an antigen presenting cell. Then, at least some of the foreign protein is proteolyzed by the cytosolic proteasome to form short peptides, which are transported into the lumen of the endoplasmic reticulum of the antigen presenting cell. There, the foreign peptides are loaded onto MHC class I molecules and transported by vesicles to the cell surface of the antigen presenting cell for recognition by CD8+ cytotoxic T cells. MHC I expression on cancer cells is required for detection and destruction by T-cells, and cytotoxic T lymphocytes (CTLs, CD8+) require tumor antigen presentation on the target cell by MHC Class I molecules to delineate self from non-self. One of the most common means by which tumors evade the host immune response is by down-regulation of MHC Class I molecule expression by tumor cells, such that the tumor has low MHCI expression, thereby rendering any endogenous or therapeutic anti-tumor T cell responses ineffective (Haworth et al., Pediatr Blood Cancer. 2015 April; 62(4): 571-576). Most often, the loss of MHC expression on tumor cells is mediated by epigenetic events and transcriptional down-regulation of the MHC locus and/or the antigen processing machinery. Lack of a processed peptide antigen leads to decreased MHC expression since empty MHC molecules are not stable on the cell surface.

A class II MHC molecule, which is present on professional antigen presenting cells, presents foreign peptides to helper T cells. Foreign peptides are endocytosed and degraded in the acidic environment of the endosome, which means that the peptides are never presented in the cytosol and remain in a subcellular compartment topologically equivalent to the extracellular space. The peptides bind to preassembled MHC class II proteins in a specialized endosomal compartment, and the loaded MHC class II molecule is then transported to the plasma membrane of the antigen presenting cell for presentation to CD4+ helper T cells. (Alberts et al. Molecular Biology of the Cell 4th Ed., Garland Science, New York (2002) p. 1407). Antigens also can be loaded onto antigen presenting cells by acquisition of MHC class II molecules from the surface of donor cells. Peptide-MHC transfer (cross-dressing”), involves generation of peptide-MHC class II complexes within the donor cell, and their subsequent transfer to recipient antigen presenting cells, which are then able to present the intact, largely unprocessed peptide-MHC class II complexes to helper T cells. (Campana, S. et al., Immunol. Letters (2015) 168(2): 349-54). Endogenous antigens can also be presented by MHC class II when they are degraded through autophagy. (Schmid, D. et al. (2007) Immunity 26(1): 79-92).

As used herein, the terms “modify” or “modulate” and their various grammatical forms is meant to refer to regulating, altering, adapting or adjusting to a certain measure or proportion. With respect to an immune response to tumor cells, these terms are meant to refer to changing the form or character of the immune response to the tumor cells via one or more recombinant DNA techniques such that the immune cells are able to recognize and kill tumor cells.

As used herein the term “natural killer (NK) cells” refers to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

As used herein, the term “natural killer T cell” or “NKT” refers to invariant natural killer T (iNKT) cells, also known as type-I NKT cells, as well as all subsets of non-invariant (Vα24- and Vα24+) natural killer T cells, which express CD3 and an αβ T cell receptor (TCR) (herein termed “natural killer αβ T cells”) or γΔ TCR (herein termed “natural killer γΔ T cells”), all of which have demonstrated capacity to respond to non-protein antigens presented by CD1 antigens. The non-invariant NKT cells encompassed by the methods of the described invention share in common with type-I NKT cells the expression of surface receptors commonly attributed to natural killer (NK) cells, as well as a TCR of either αβ or γΔ TCR gene locus rearrangement/recombination.

As used herein, the term “invariant natural killer T cell” is used interchangeably with the term “iNKT,” and is meant to refer to a subset of T-cell receptor (TCR)α-expressing cells that express a restricted TCR repertoire that, in humans, is composed of a Vα24-Jα18 TCRα chain, which is, for example, coupled with a Vβ11 TCRβ chain. It encompasses all subsets of CD3+Vα24+Vβ11+type-I NKT cells (CD3+CD4+CD8-Vα24+Vβ11+, CD3+CD4-CD8+Vα24+Vβ11+, and CD3+CD4-CD8-Vα24+Vβ11+) as well as those cells, which can be confirmed to be type-I NKT cells by gene expression or other immune profiling, but have down-regulated surface expression of Vα24 (CD3+Vα24-). This includes cells which either do or do not express the regulatory transcription factor FOXP3. Unlike conventional T cells, which mostly recognize peptide antigens presented by MHC molecules, iNKT cells recognize glycolipid antigens presented by the non-polymorphic MHC class 1-like CD1d.

As used herein, the term “pattern recognition receptors” or “PRRs” refers to receptors that are present at the cell surface to recognize extracellular pathogens; in the endosomes where they sense intracellular invaders, and finally in the cytoplasm. They recognize conserved molecular structures of pathogens, called pathogen associated molecular patterns (PAMPs) specific to the microorganism and essential for its viability. PRRs are divided into four families: toll-like receptors (TLR); nucleotide oligomerization receptors (NLR); C-type leptin receptors (CLR), and RIG-1 like receptors (RLR).

As used herein, the term “NKT cells” refers to a population of cells that includes CD3+Vα24+ NKT cells, CD3+Vα24− NKT cells, CD3+Vα24− CD56+ NKT cells, CD3+Vα24-CD161+ NKT cells, CD3+γδ-TCR+ T cells, and mixtures thereof.

As used herein, the term “nonexpanded” is meant to refer to a cell population that has not been grown in culture (in vitro) to increase the number of cells in the cell population.

As used herein, the term “overall survival” (OS) is meant to refer to the length of time from either the date of diagnosis or the start of treatment for a disease, such as cancer, that patients diagnosed with the disease are still alive.

As used herein, the term “parenteral” and its other grammatical forms is meant to refer to administration of a substance occurring in the body other than by the mouth or alimentary canal. For example, the term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), or infusion techniques.

As used herein, the term “perforin” is meant to refer to a molecule that can insert into the membrane of target cells and promote lysis of those target cells. Perforin-mediated lysis is enhanced by enzymes called granzymes.

As used herein, a “peripheral blood mononuclear cell” or “PBMC” refers to an immune cell with a round nucleus found in peripheral blood that remains at the less dense, upper interface of the Ficoll layer, often referred to as the buffy coat, and are the cells collected when the Ficoll fractionation method is used. These cells consist of lymphocytes (T cells, B cells, NK cells) and monocytes. In humans, lymphocytes make up the majority of the PBMC population, followed by monocytes, and only a small percentage of dendritic cells.

As used herein, the term “pharmaceutical composition” is meant to refer to a composition comprising an active ingredient and a pharmaceutically acceptable carrier that is employed to prevent, reduce in intensity, cure or otherwise treat a target condition, syndrome, disorder or disease.

As used herein, the term “pharmaceutically acceptable carrier” is meant to refer to any substantially non-toxic carrier conventionally useable for administration of pharmaceuticals in which the cell product of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

As used herein, the term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydro iodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids.

As used herein, the terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. According to some embodiments, the peptide is of any length or size.

As used herein, the term “purify” is meant to refer to freeing from extraneous or undesirable elements.

As used herein, the term “recurrence” with respect to cancer is meant to refer to a cancer that has recurred (come back), usually after a period of time during which the cancer could not be detected. The cancer may come back to the same place as the original (primary) tumor or to another place in the body.

As used herein, the term “resistant cancer” is meant to refer to a cancer that does not respond to a treatment at the beginning of such treatment or sometime during such treatment.

As used herein, the term “secretion” and its various grammatical forms is meant to refer to production by a cell of a physiologically active substance and its movement out of the cell in which it is formed.

As used herein, the term “stimulate” in any of its grammatical forms as used herein is meant to refer to inducing activation or increasing activity.

As used herein, the term “sufficient to stimulate NKT cell expansion” refers to an amount or level of a signaling event or stimulus, e.g. an amount of alpha-galactosylceramide (αGalCer), or an analog or functional equivalent thereof, that promotes preferential expansion of a type-I NKT cell.

As used herein, the term “sufficient to stimulate NKT cell activation” refers to an amount or level of a signaling event or stimulus, e.g. an amount of IL-2, IL-7, IL-15 and IL-12, that promotes cytokine secretion or cell-killing activity of a type-I NKT cell.

As used herein, the terms “subject” or “individual” or “patient” are used interchangeably to refer to a member of an animal species of mammalian origin, including humans.

As used herein, the phrase “subject in need thereof” is meant to refer to a patient that (i) will be administered an immunogenic composition (e.g. a population of type-I NKT cells) according to the described invention, (ii) is receiving an immunogenic composition (e.g. a population of type-I NKT cells) according to the described invention; or (iii) has received an immunogenic composition (e.g. a population of type-I NKT cells) according to the described invention, unless the context and usage of the phrase indicates otherwise.

As used herein, the term “superactivated cytokine killer T cells” (or SCKTCs) refers to cells derived from cytokine killer T cells (CKTCs) by contacting CKTCs in vitro with cytokines IL-2, IL-7, IL-15 and IL-12 in a predetermined order and time of addition.

As used herein, the term “T cell receptor” (TCR) is meant to refer to a complex of integral membrane proteins that participate in the activation of T cells in response to an antigen. The TCR expressed by the majority of T cells consisting of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions.

Naive conventional CD4 T cells can differentiate into four distinct T cell populations, a process that is determined by the pattern of signals they receive during their initial interaction with antigen. These 4 T cell populations are Th1, Th2, Th17, and induced regulatory T (iTreg) cells. Th1 cells, which are effective inducers of cellular immune responses, mediate immune responses against intracellular pathogens, and are responsible for the induction of some autoimmune diseases. Their principal cytokine products are IFNγ (which enhances several mechanisms important in activating macrophages to increase their microbiocidal activity), lymphotoxin α (LTα), and IL-2, which is important for CD4 T cell memory. Th2 cells, which are effective in helping B cells develop into antibody producing cells, mediate host defense against extracellular parasites, are important in the induction and persistence of asthma and other allergic disease, and produce IL-4, IL-5, IL-9, IL-10 (which suppresses Th1 cell proliferation and can suppress dendritic cell function), IL-13, IL-25 (signaling through IL-17RB, enhances the production of IL-4, IL-5, and IL-13 by a c-kit-FcεRI-nonlymphocyte population, serves as an initiation factor as well as an amplification factor for Th2 responses) and amphiregulin. IL-4 and IL-10 produced by Th2 cells block IFNγ production by Th1 cells. Th17 cells produce IL-17a, IL-17f, IL-21, and IL-22. IL-17a can induce many inflammatory cytokines, IL6 as well as chemokines such as IL-8 and plays an important role in inducing inflammatory responses. Treg cells play a critical role in maintaining self-tolerance and in regulating immune responses. They exert their suppressive function through several mechanisms, some of which require cell-cell contact. The molecular basis of suppression in some cases is through their production of cytokines, including TGFβ, IL-10, and IL-35. TGFβ produced by T reg cells may also result in the induction if iTreg cells from naïve CD4 T cells. CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. Zhu, J. and Paul, WE, Blood (2008) 112: 1557-69). Resting naïve CD8+ T cells, when primed by antigen presenting cells that have acquired antigens from the infected macrophages through direct infection or cross-presentation in secondary lymphoid organs, such as lymph nodes and spleen, react to pathogens by massive expansion and differentiation into cytotoxic T lymphocyte effector cells that migrate to all corners of the body to clear the infection. In the majority of viral infections, however, CD8 T cell activation requires CD4 effector T cell help to activate dendritic cells for them to become able to stimulate a complete CD8 T cell response. CD4 T cells that recognize related antigens presented by the APC can amplify the activation of naïve CD8 T cells by further activating the APC. B7 expressed by the dendritic cell first activates the CD4 T cells to express IL-2 and CD40 ligand. CD40 ligand binds CD40 on the dendritic cell, delivering an additional signal that increases the expression of B7 and 4-1BBL by the dendritic cell, which in turn provides additional co-stimulation to the naïve CD8 T cell. The IL-2 produced by activated CD4 T cells also acts to promote effector CD T cell differentiation.

The CD3 (TCR complex) is a protein complex composed of four distinct chains. In mammals, the complex contains a CD3γ chain, a CD3δ chain, and two CD3ε chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

As used herein, the term “therapeutic agent” is meant to refer to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

As used herein, the term “therapeutic amount”, “therapeutically effective amount”, an “amount effective”, or “pharmaceutically effective amount” of an active agent is used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein, the term “therapeutic effect” is meant to refer to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

As used herein, the term “treating” includes abrogating, substantially inhibiting, slowing or reversing the progression of a condition, substantially ameliorating clinical symptoms of a condition, or substantially preventing the appearance of clinical symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

In accordance with the described invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A practical Guide To Molecular Cloning (1984); F. M. Ausubel et al. (eds.), Current Protocols in Molecular Biology, John Wiley & Sons, Inc. (1994); among others.

### Methods for Preparing a Pharmaceutical Composition Comprising a Cell Product Comprising an Expanded, and Enriched Population of Superactivated Cytokine Killer T Cells (SCKTCS)

According to one aspect, the present disclosure describes a method for preparing a pharmaceutical composition comprising an enriched population of superactivated cytokine killer T cells (SCKTCs) comprising, in order

(a) isolating a population of mononuclear cells (MCs) comprising a population of cytokine killer T cells (CKTCs);

(b) optionally transporting the preparation of (a) to a processing facility under sterile conditions;

(c) culturing the population of MCs in a culture system;

(d) contacting the culture system of step (c) with alpha-galactosylceramide (αGalCer), or an analog or functional equivalent thereof; a first population of cells comprising CD1d and αGalCer, or an analog or functional equivalent thereof, or both wherein the contacting is sufficient to stimulate CKTC expansion;

(e) contacting the culture system of step (d) with IL-2, IL-7, IL-15 and IL-12, in a predetermined order and time of addition, wherein the contacting is sufficient to stimulate CKTC activation and to form the enriched population of SCKTC cells;

(f) collecting the enriched population of SCKTC cells from the culture system to form a SCKTC cell product; wherein the enriched population of SCKTCs of (f) is characterized by one or more of an improved ability to secrete effector cytokines or an improved cytotoxicity compared to the population of CKTCs of (a); and

(g) formulating the cell product with a pharmaceutically acceptable carrier to form the pharmaceutical composition.

According to some embodiments, a source of the mononuclear cells is blood. According to some such embodiments, the blood is peripheral blood and the MCs are peripheral blood MCs (PBMCs). According to some embodiments, the PBMCs are derived from a human subject. According to some embodiments, the MCs are isolated from a Ficoll-Paque gradient fraction.

According to some embodiments, the culturing in (c) is for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days, or more. According to some embodiments, the culturing in (c) is for a time effective for adherence of at least some of the CTKCs to a surface of the culture system. According to some embodiments, step (c) optionally comprises re-suspending the MCs and adjusting the concentration of MCs to a range of about 5×10⁵ cells/ml to about 3×10⁶ cells/ml, inclusive, before performing step (d). According to one embodiment, step (c) optionally comprises re-suspending the MCs and adjusting the concentration of MCs to about 5×10⁵ cells/ml, about 5.1×10⁵ cells/ml, about 5.2×10⁵ cells/ml, about 5.3×10⁵ cells/ml, about 5.4×10⁵ cells/ml, about 5.5×10⁵ cells/ml, about 5.6×10⁵ cells/ml, about 5.7×10⁵ cells/ml, about 5.8×10⁵ cells/ml, about 5.9×10⁵ cells/ml, about 6×10⁵ cells/ml, about 6.1×10⁵ cells/ml, about 6.2×10⁵ cells/ml, about 6.3×10⁵ cells/ml, about 6.4×10⁵ cells/ml, about 6.5×10⁵ cells/ml, about 6.6×10⁵ cells/ml, about 6.7×10⁵ cells/ml, about 6.8×10⁵ cells/ml, about 6.9×10⁵ cells/ml, about 7×10⁵ cells/ml, about 7.1×10⁵ cells/ml, about 7.2×10⁵ cells/ml, about 7.3×10⁵ cells/ml, about 7.4×10⁵ cells/ml, about 7.5×10⁵ cells/ml, about 7.6×10⁵ cells/ml, about 7.7×10⁵ cells/ml, about 7.8×10⁵ cells/ml, about 7.9×10⁵ cells/ml, about 8×10⁵ cells/ml, about 8.1×10⁵ cells/ml, about 8.2×10⁵ cells/ml, about 8.3×10⁵ cells/ml, about 8.4×10⁵ cells/ml, about 8.5×10⁵ cells/ml, about 8.6×10⁵ cells/ml, about 8.7×10⁵ cells/ml, about 8.8×10⁵ cells/ml, about 8.9×10⁵ cells/ml, about 9×10⁵ cells/ml, about 9.1×10⁵ cells/ml, about 9.2×10⁵ cells/ml, about 9.3×10⁵ cells/ml, about 9.4×10⁵ cells/ml, about 9.5×10⁵ cells/ml, about 9.6×10⁵ cells/ml, about 9.7×10⁵ cells/ml, about 9.8×10⁵ cells/ml, about 9.9×10⁵ cells/ml, about 1×10⁶ cells/ml, about 1.1×10⁵ cells/ml, about 1.2×10⁵ cells/ml, about 1.3×10⁵ cells/ml, about 1.4×10⁵ cells/ml, about 1.5×10⁶ cells/ml, about 1.6×10⁵ cells/ml, about 1.7×10⁵ cells/ml, about 1.8×10⁵ cells/ml, 1.9×10⁵ cells/ml, about 2×10⁶ cells/ml, about 2.1×10⁵ cells/ml, about 2.2×10⁵ cells/ml, about 2.3×10⁵ cells/ml, 2.4×10⁵ cells/ml, about 2.5×10⁶ cells/ml, about 2.6×10⁵ cells/ml, about 2.7×10⁵ cells/ml, 2.8×10⁵ cells/ml, 2.9×10⁵ cells/ml, or about 3×10⁶ cells/ml before performing step (c).

According to some embodiments, t the αGalCer, or an analog or functional equivalent thereof, is OCH. According to one embodiment, the αGalCer, or an analog or functional equivalent thereof, is an α-GalCer analog of structural formula:

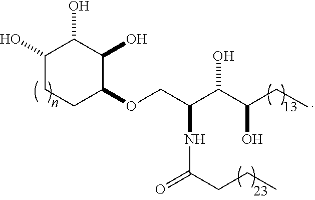

According to some embodiments, the αGalCer, or an analog or functional equivalent thereof is maintained at a constant concentration from step (c) to step (f). In a further embodiment, the concentration of αGalCer, or an analog or functional equivalent thereof, ranges from about 50 ng/ml to about 500 ng/ml, from about 100 ng/ml to about 500 ng/ml, from about 150 ng/ml to about 500 ng/ml, from about 200 ng/ml to about 500 ng/ml, from about 250 ng/ml to about 500 ng/ml, from about 300 ng/ml to about 500 ng/ml, from about 350 ng/ml to about 500 ng/ml, from about 400 ng/ml to about 500 ng/ml, or from about 450 ng/ml to about 500 ng/ml. According to some embodiments, the concentration of αGalCer, or an analog or functional equivalent thereof, is maintained at a concentration of about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 210 ng/ml, about 220 ng/ml, about 230 ng/ml, about 240 ng/ml, about 250 ng/ml, about 260 ng/ml, about 270 ng/ml, about 280 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, about 440 ng/ml, about 450 ng/ml, about 460 ng/ml, about 470 ng/ml, about 480 ng/ml, about 490 ng/ml, or about 500 ng/ml.

According to some embodiments of the methods describe herein, IL-2 is maintained at a constant concentration from step (e) to step (f). According to some embodiments, the concentration of IL-2 is between about 10 U/ml to about 100 U/ml, for example between about 10 U/ml to about 100 U/ml, about 15 U/ml to about 100 U/ml, about 20 U/ml to about 100 U/ml, about 25 U/ml to about 100 U/ml, about 30 U/ml to about 100 U/ml, about 35 U/ml to about 100 U/ml, about 40 U/ml to about 100 U/ml, about 45 U/ml to about 100 U/ml, about 50 U/ml to about 100 U/ml, about 55 U/ml to about 100 U/ml, about 60 U/ml to about 100 U/ml, about 65 U/ml to about 100 U/ml, about 70 U/ml to about 100 U/ml, about 75 U/ml to about 100 U/ml, about 80 U/ml to about 100 U/ml, about 85 U/ml to about 100 U/ml, about 90 U/ml to about 100 U/ml, or about 95 U/ml to about 100 U/ml. According to some embodiments, the concentration of IL-2 is about 10 U/ml, about 15 U/ml, about 20 U/ml, about 25 U/ml, about 30 U/ml, about 35 U/ml, about 40 U/ml, about 45 U/ml, about 50 U/ml, about 55 U/ml, about 60 U/ml, about 65 U/ml, about 70 U/ml, about 75 U/ml, about 80 U/ml, about 85 U/ml, about 90 U/ml, about 95 U/ml, or about 100 U/ml.

According to some embodiments of the methods describe herein, IL-7 is maintained at a constant concentration from step (e) to step (f). According to some embodiments, the concentration of IL-7 is between about 10 ng/ml to about 200 ng/ml, for example between about 10 ng/ml to about 200 ng/ml, about 20 ng/ml to about 200 ng/ml, about 30 ng/ml to about 200 ng/ml, about 40 ng/ml to about 200 ng/ml, about 50 ng/ml to about 200 ng/ml, about 60 ng/ml to about 200 ng/ml, about 70 ng/ml to about 200 ng/ml, about 80 ng/ml to about 200 ng/ml, about 90 ng/ml to about 200 ng/ml, about 100 ng/ml to about 200 ng/ml, about 110 ng/ml to about 200 ng/ml, about 120 ng/ml to about 200 ng/ml, about 130 ng/ml to about 200 ng/ml, about 140 ng/ml to about 200 ng/ml, about 150 ng/ml to about 200 ng/ml, about 160 ng/ml to about 200 ng/ml, about 170 ng/ml to about 200 ng/ml, about 180 ng/ml to about 200 ng/ml, or about 190 ng/ml to about 200 ng/ml. According to some embodiments, the concentration of IL-7 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 110 ng/ml, about 15 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml.

According to some embodiments, IL-2 is added in step (e) at between about day 6 and day 8 of culture. According to some embodiments, IL-2 is added in step (e) at about day 6 of culture. According to some embodiments, IL-2 is added in step (e) at about day 7 of culture. According to some embodiments, IL-2 is added in step (e) at about day 8 of culture.

According to some embodiments, IL-7 is added in step (e) at between about day 6 and day 8 of culture. According to some embodiments, IL-7 is added in step (e) at about day 6 of culture. According to some embodiments, IL-7 is added in step (e) at about day 7 of culture. According to some embodiments, IL-7 is added in step (e) at about day 8 of culture.

According to some embodiments, IL-2 and IL-7 are added simultaneously. According to some embodiments, IL-2 and IL-7 are added simultaneously at day 7.

According to some embodiments, IL-15 is added in step (e) at between about day 13 and day 15 of culture. According to some embodiments, IL-15 is added in step (e) at about day 13 of culture. According to some embodiments, IL-15 is added in step (e) at about day 14 of culture. According to some embodiments, IL-15 is added in step (e) at about day 15 of culture.

According to some embodiments, IL-15 is added in step (e) at between about day 19 and day 21 of culture. According to some embodiments, IL-12 is added in step (e) at about day 19 of culture. According to some embodiments, IL-12 is added in step (e) at about day 20 of culture. According to some embodiments, IL-12 is added in step (e) at about day 21 of culture.

According to some embodiments, step (f) is carried out at least at day 21. According to some embodiments, step (f) is carried out at day 21. According to some embodiments, step (f) is carried out at day 22. According to some embodiments, step (f) is carried out at day 23. According to some embodiments, step (f) is carried out at day 24.

According to some embodiments of the methods describe herein, IL-15 is maintained at a constant concentration from step (e) to step (f). According to some embodiments, the concentration of IL-15 is between about 10 ng/ml to about 100 ng/ml, for example between about 10 ng/ml to about 100 ng/ml, about 15 ng/ml to about 100 ng/ml, about 20 ng/ml to about 100 ng/ml, about 25 ng/ml to about 100 ng/ml, about 30 ng/ml to about 100 ng/ml, about 35 ng/ml to about 100 ng/ml, about 40 ng/ml to about 100 ng/ml, about 45 ng/ml to about 100 ng/ml, about 50 ng/ml to about 100 ng/ml, about 55 ng/ml to about 100 ng/ml, about 60 ng/ml to about 100 ng/ml, about 65 ng/ml to about 100 ng/ml, about 70 ng/ml to about 100 ng/ml, about 75 ng/ml to about 100 ng/ml, about 80 ng/ml to about 100 ng/ml, about 85 ng/ml to about 100 ng/ml, about 90 ng/ml to about 100 ng/ml, or about 95 ng/ml to about 100 ng/ml. According to some embodiments, the concentration of IL-15 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml.

According to some embodiments of the methods describe herein, IL-12 is maintained at a constant concentration from step (e) to step (0.

According to some embodiments, the method further comprises a step between steps (e) and (f) of transporting the culture from the processing facility to a treatment facility. According to some embodiments, the transporting step is initiated within at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours of the addition of IL-12.

According to some embodiments, the concentration of IL-12 is between about 10 ng/ml to about 100 ng/ml, for example between about 10 ng/ml to about 100 ng/ml, about 15 ng/ml to about 100 ng/ml, about 20 ng/ml to about 100 ng/ml, about 25 ng/ml to about 100 ng/ml, about 30 ng/ml to about 100 ng/ml, about 35 ng/ml to about 100 ng/ml, about 40 ng/ml to about 100 ng/ml, about 45 ng/ml to about 100 ng/ml, about 50 ng/ml to about 100 ng/ml, about 55 ng/ml to about 100 ng/ml, about 60 ng/ml to about 100 ng/ml, about 65 ng/ml to about 100 ng/ml, about 70 ng/ml to about 100 ng/ml, about 75 ng/ml to about 100 ng/ml, about 80 ng/ml to about 100 ng/ml, about 85 ng/ml to about 100 ng/ml, about 90 ng/ml to about 100 ng/ml, or about 95 ng/ml to about 100 ng/ml. According to some embodiments, the concentration of IL-12 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml.

According to some embodiments, the method further comprises a step of replenishing the culture medium in the culture system every 2 to 3 days. According to some embodiments, the replenishing step includes adding pulses of fresh dendritic cells loaded αGalCer or an analog or functional equivalent thereof to the culture system. According to some embodiments, the number of pulses of the fresh population of cells comprising CD and αGalCer is at least 1, at least 2, at least 3, at least 4, at least 5, at least 6, at least 7, at least 8, at least 9, or at least 10.

According to some embodiments, steps (c)-(f) are carried out in a culture medium selected from X-VIVO-15 serum-free medium, and RPMI 1640 medium containing 10% of either fetal bovine serum (FBS) or 10% autologous serum.

### Antigen Presenting Cells

According to some embodiments, the cell comprising CD1d and alpha-galactosylceramide (αGalCer) is an antigen presenting cell. An antigen presenting cell is a class of cells capable of displaying on its surface one or more antigens in the form of a peptide-MHC complex recognizable by specific effector cells of the immune system, and thereby inducing an effective cellular immune response against the antigen or antigens being presented. Examples of professional APCs are dendritic cells and macrophages, although any cell expressing MHC Class I molecules or MHC Class II molecules can potentially present peptide antigen. According to some embodiments, an APC can be a cell or population of cells that is engineered to present one or more antigens (i.e. an artificial APC (aAPC).

According to some embodiments, the antigen presenting cell is a dendritic cell (DC). According to some embodiments, the dendritic cell is loaded with αGalCer. According to another embodiment, the dendritic cell loaded with αGalCer is derived from the MCs and is an adherent cell. According to another embodiment in the method for preparing the dendritic cell loaded with αGalCer, the dendritic cell loaded with αGalCer is an adherent cell.

According to some embodiments, the dendritic cell loaded with αGalCer is prepared by a method comprising (a) isolating a population of mononuclear cells (MCs); (b) culturing the population of MCs in a culture system; (c) contacting the culture system with IL-4 and GM-CSF, wherein the contacting is sufficient to induce differentiation of the MCs into dendritic cells; (d) contacting the culture system with αGalCer, wherein the contacting is sufficient to load the dendritic cells with αGalCer.

According to some embodiments of the method for preparing dendritic cells loaded with αGalCer, the population of MCs when the cultures are initiated comprises between about 1×10⁵ cells/ml and about 5×10⁶ cells/ml. According to some embodiments, the population of MCs is about 1×10⁵ cells/ml, about 1.5×10⁵ cells/ml, about 1×10⁵ cells/ml, about 1.5×10⁵ cells/ml, about 3×10⁵ cells/ml, about 3.5×10⁵ cells/ml, about 4×10⁵ cells/ml, about 4.5×10⁵ cells/ml, about 5×10⁵ cells/ml, about 5.5×10⁵ cells/ml, about 6×10⁵ cells/ml, about 6.5×10⁵ cells/ml, about 7×10⁵ cells/ml, about 7.5×10⁵ cells/ml, about 8×10⁵ cells/ml, about 8.5×10⁵ cells/ml, about 9×10⁵ cells/ml, about 9.5×10⁵ cells/ml, about 1×10⁶ cells/ml, about 1.5×10⁶ cells/ml, about 2×10⁶ cells/ml, about 2.5×10⁶ cells/ml, about 3×10⁶ cells/ml, about 3.5×10⁶ cells/ml, about 4×10⁶ cells/ml, about 4.5×10⁶ cells/ml, or about 5×10⁶ cells/ml.

According to one embodiment, the concentration of IL-4 in step (c) is about 500 U/ml. According to one embodiment, the concentration of IL-4 is between about 400-600 U/ml, for example about 400 U/ml, about 450 U/ml, about 500 U/ml, about 550 U/ml, about 600 U/ml. According to one embodiment, the concentration of GM-CSF in step (c) is about 50 ng/ml. According to one embodiment, the concentration of GM-CSF in step (c) is between about 40-60 ng/ml, for example about 40 ng/ml, about 45 ng/ml, about 50 ng/ml, about 55 ng/ml or about 60 ng/ml. According to one embodiment, step (d) is carried out from about 5 days to about 7 days after step (b). According to one embodiment, step (d) is carried out at about 5 days after step (b). According to one embodiment, step (d) is carried out at about 6 days after step (b). According to one embodiment, step (d) is carried out at about 7 days after step (b).

According to some embodiments, steps (c)-(e) are carried out in a culture medium selected from RPMI 1640 medium containing 10% FBS or autologous serum.

Stimulation of the CKTCs with Alpha-Galactosylceramide (αGalCer) and Analogs

Upon primary stimulation, in particular in response to an α-galactosylceramide (α-GalCer) by a nonmammalian glycosphingolipid (GSL), type-I NKT cells produce large amounts of interferon (IFN)-γ and interleukin (IL)-4, that leads to downstream activation of DCs, NK cells, B cells, and conventional T cells. α-GalCer, also known as KRN7000, is a simplified glycolipid analogue of agelasphin, which was originally isolated from a marine sponge Agelas mauritianus (Kobayahi et al., Oncol Res. 1995; 7(10-11):529). α-GalCer is composed of an α-linked galactose, a phytosphingosine and an acyl chain. Recognition of the α-GalCer-CD1d complex by the type-I NKT cell TCR results in the secretion of a range of cytokines, and the initiation of a powerful immune response. OCH, an α-GalCer analogue with a shorter phytosphingosine chain, stimulates type-I NKT cells to secrete higher amounts of IL-4 than IFN-γ, triggering the immune response toward Th2 (Journal of Biomedical Science 2017, 24:22). Synthetic glycolipids or α-GalCer analogs chemically modified to induce more precise and predictable cytokine profile than α-GalCer have been synthesized and tested. Hung, J-T et al. (Journal of Biomedical Science (2017) 24:22), incorporated by reference in its entirety herein, describes a number of α-GalCer analogues. U.S. Pat. No. 9,365,496, incorporated by reference in its entirety herein, also describes various α-GalCer analogs with the structural formula:

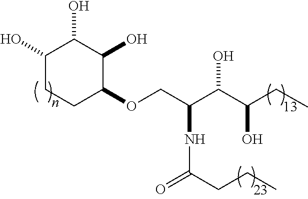

Another class of type-I NKT cell agonist, β-ManCer has been described (O'Konek et al., J Clin Invest. 2011 February; 121(2):683-94). This compound has an identical ceramide structure to that of α-GalCer (KRN7000), which contributes to the binding with CD1d, with a beta-linked mannose instead of alpha-linked galactose. It had been believed in the field that the alpha-linked sugar moiety was a critical feature of α-GalCer to elicit tumor immunity. Therefore, the discovery of relatively strong anti-tumor activity of β-ManCer was unexpected. While the protection induced by β-ManCer was type-I NKT cell-dependent, the protection was independent of IFN-γ but dependent on TNF-α and nitric oxide synthase (NOS). Furthermore, consistent with the distinct mechanism of protection, α-GalCer and β-ManCer synergized to induce tumor immunity when suboptimal doses were used. In addition, β-ManCer has much weaker ability to induce long-term anergy in type-I NKT cells than α-GalCer (O'Konek et al, Clin Cancer Res. 2013 Aug. 15; 19(16):4404-11). Similar to α-GalCer, β-ManCer can enhance the effect of a tumor vaccine (Mattarollo et al., Blood. 2012 Oct. 11; 120(15):3019-29). Thus, type-I NKT cells can use multiple pathways/mechanisms dependent on the antigens that they recognize.

According to some embodiments, the population of CKTCs of the described invention comprises a subpopulation of CD3+ T cells. According to some embodiments, the population of CKTCs comprises a subpopulation of NKT cells. According to one embodiment, the subpopulation of NKT cells comprises CD3+Vα24+ cells. According to one embodiment, the subpopulation of NKT cells comprises CD3+Vα24− cells. According to one embodiment, the subpopulation of NKT cells comprises CD3+CD56+ cells. According to some embodiments, the subpopulation of NKT cells comprise a subpopulation of type 1 NKT cells. According to some embodiments, the T cell receptor of the subpopulation of NKT cells comprises a Vα24-Jα18 TCRα chain. According to some embodiments, the T cell receptor of the subpopulation of NKT cells comprises a Vα24-Jα18 TCRα chain and a Vβ11 β chain. According to some embodiments, the subpopulation of NKT cells recognize glycolipid antigens presented by CD1d. According to some embodiments, the glycolipid antigen is αGalCer or an analog or functional equivalent thereof.

### CKTC Expansion and Activation

When type-I NKT cells are stimulated with α-GalCer, they produce IFN-γ. Simultaneously, they activate antigen-presenting cells (APCs) through CD40-CD40L interaction, especially inducing DCs to mature and up-regulate co-stimulatory receptors such as CD80 and CD86. DCs also produce IL-12 upon their interaction with type-I NKT cells. IL-12 induces more IFN-γ production by other T cells and plays a critical role together with IFN-γ in the activation of downstream effectors such as NK cells, CD8+ T cells and γδ T cells (Paget et al., J Immunol. 2012 Apr. 15; 188(8):3928-39). The interaction of type-I NKT cells with APCs offers activation signals to (i.e., licenses) APCs to render them able to cross-prime to CD8+ T cells through the induction of CD70 and CCL17 (Taraban et al., J Immunol. 2008 Apr. 1; 180(7):4615-20; Fujii et al., Immunol Rev. 2007 December; 2200:183-98).

According to some embodiments, the activating of the population of CKTCs can comprise one or more of inducing secretion of a cytokine by the population of CKTCs, stimulating proliferation of the population of CKTCs, or modulating expression of one or more markers on the cell surface of the CKTCs. According to some embodiments, the cytokine whose expression is modulated is one or more selected from the group consisting of IFNγ, IL-4, IL-5, IL-6, or IL-10.

Activation and expansion of the population of CKTCs can be measured by various assays as described herein. Exemplary activities that may be measured include the induction of proliferation, the induction of expression of activation markers in the population of CKTCs, the induction of cytokine secretion by the population of CKTCs, the induction of signaling in the population of CKTCs, and an increase in the cytotoxic activity of the population of CKTCs.

### Cytokine Secretion

The activation of CKTCs to form SCKTCs may be assessed or measured by determining secretion of cytokines, including one or more of gamma interferon (IFNγ), interleukin 4 (IL-4), interleukin 5 (IL-5), interleukin 6 (IL-6) or interleukin-10 (IL-10). According to some embodiments, ELISA is used to determine cytokine secretion, for example secretion of gamma interferon (IFNγ), IL-4, IL-5, IL-6 or IL-10. The ELISPOT (enzyme-linked immunospot) technique may be used to detect CKTCs and SCKTCs that secrete a given cytokine (e.g., gamma interferon (IFNγ)) in response to the methods described herein. For example, a culture system can be set up whereby a population of CKTCs or SCKTCs produced by the methods described herein are cultured within wells that have been coated with anti-IFNγ antibodies. The secreted IFNγ is captured by the coated antibody and then revealed with a second antibody coupled to a chromogenic substrate. Locally secreted cytokine molecules form spots, with each spot corresponding to one IFNγ-secreting cell. The number of spots allows one to determine the frequency of IFNγ-secreting cells in the analyzed sample. The ELISPOT assay has also been described for the detection of tumor necrosis factor alpha (TNFα), IL-4, IL-5, IL-6, IL-10, IL-12, granulocyte-macrophage colony-stimulating factor (GM-CSF), and granzyme B-secreting lymphocytes (Klinman D, Nutman T Current protocols in immunology. New York, N.Y: John Wiley & Sons, Inc.; 1994. pp. 6.19.1-6.19.8, incorporated by reference in its entirety herein).

Flow cytometric analyses of intracellular cytokines may be used to measure the cytokine content in culture supernatants, but provide no information on the number of NKT cells that actually secrete the cytokine. When lymphocytes are treated with inhibitors of secretion, such as monensin or brefeldin A, they accumulate cytokines within their cytoplasm upon activation. After fixation and permeabilization, intracellular cytokines can be quantified by cytometry. This technique allows the determination of the cytokines produced, the type of cells that produce these cytokines, and the quantity of cytokine produced per cell.

According to some embodiments, cytokine production by the enriched population of SCKTCs is characterized as IL-4 low, IL-5 low, IL-6 low, IL-10 low, IFNγ high.

According to one embodiment, the amount of IFN-γ produced by the population of cells is about 5000 pg/ml or greater.

According to some embodiments, the amount of IL-4 produced by the population of cells is about 5 pg/ml. According to one embodiment, the amount of IL-4 produced by the population of cells is about 4.5 pg/ml. According to one embodiment, the amount of IL-4 produced by the population of cells is about 4 pg/ml. According to one embodiment, the amount of IL-4 produced by the population of cells is about 3.5 pg/ml. According to one embodiment, the amount of IL-4 produced by the population of cells is about 3 pg/ml. According to one embodiment, the amount of IL-4 produced by the population of cells is about 2.5 pg/ml. According to one embodiment, the amount of IL-4 produced by the population of cells is about 2 pg/ml. According to one embodiment, the amount of IL-4 produced by the population of cells is about 1.5 pg/ml. According to one embodiment, the amount of IL-4 produced by the population of cells is about 1 pg/ml. According to one embodiment, the amount of IL-4 produced by the population of cells is between about 1.0 and about 5 pg/ml. According to one embodiment, the amount of IL-4 produced by the population of cells is between about 1.5 and about 5 pg/ml. According to one embodiment, the amount of IL-4 produced by the population of cells is between about 2.0 and about 5 pg/ml. According to one embodiment, the amount of IL-4 produced by the population of cells is between about 2.5 and about 5 pg/ml. According to one embodiment, the amount of IL-4 produced by the population of cells is between about 3.0 and about 5 pg/ml. According to one embodiment, the amount of IL-4 produced by the population of cells is between about 3.5 and about 5 pg/ml. According to one embodiment, the amount of IL-4 produced by the population of cells is between about 4.0 and about 5 pg/ml. According to one embodiment, the amount of IL-4 produced by the population of cells is between about 4.5 and about 5 pg/ml.

According to some embodiments, the ratio of IFNγ to IL-4 is an indicator of one or more T cell effector functions (such as cell killing and cell activation), of the CKTCs and SCKTCs. According to some embodiments, the method is effective for achieving an IFN gamma:IL4 ratio of at least 1000, a killing rate increased at least 1.5 fold over control CTKC cells, or both.

According to one embodiment, the ratio of IFN-γ:IL-4 in culture supernatants is equal to or greater than 1000. According to one embodiment, the ratio of IFN-γ:IL-4 in culture supernatants is equal to or greater than 1200. According to one embodiment, the ratio of IFN-γ:IL-4 in culture supernatants is equal to or greater than 1300. According to one embodiment, the ratio of IFN-γ:IL-4 in culture supernatants is equal to or greater than 1400. According to one embodiment, the ratio in culture supernatants of IFN-γ:IL-4 is equal to or greater than 1500. According to one embodiment, the ratio of IFN-γ:IL-4 in culture supernatants is equal to or greater than 1550. According to one embodiment, the ratio of IFN-γ:IL-4 in culture supernatants is equal to or greater than 1600. According to one embodiment, the ratio of IFN-γ:IL-4 in culture supernatants is greater than 1650. According to one embodiment, the ratio of IFN-γ:IL-4 in culture supernatants is equal to or greater than 1700. According to one embodiment, the ratio of IFN-γ:IL-4 in culture supernatants is equal to or greater than 1750. According to one embodiment, the ratio of IFN-γ:IL-4 in culture supernatants is equal to or greater than 1800. According to one embodiment, the ratio of IFN-γ:IL-4 in culture supernatants is equal to or greater than 1850. According to one embodiment, the ratio of IFN-γ:IL-4 in culture supernatants is equal to or greater than 1900. According to one embodiment, the ratio of IFN-γ:IL-4 in culture supernatants is greater than 1950. According to one embodiment, the ratio of IFN-γ:IL-4 in culture supernatants is equal to or greater than 2000. According to one embodiment, the ratio of IFN-γ:IL-4 is equal to or greater than 2050. According to one embodiment, the ratio of IFN-γ:IL-4 in culture supernatants is equal to or greater than 2100. According to one embodiment, the ratio of IFN-γ:IL-4 in culture supernatants is equal to or greater than 2150. According to one embodiment, the ratio of IFN-γ:IL-4 in culture supernatants is equal to or greater than 2200. According to one embodiment, the ratio of IFN-γ:IL-4 in culture supernatants is equal to or greater than 2250. According to one embodiment, the ratio of IFN-γ:IL-4 in culture supernatants is equal to or greater than 2300. According to one embodiment, the ratio of IFN-γ:IL-4 in culture supernatants is equal to or greater than 2350. According to one embodiment, the ratio of IFN-γ:IL-4 in culture supernatants is equal to or greater than 2400. According to one embodiment, the ratio of IFN-γ:IL-4 in culture supernatants is equal to or greater than 2450. According to one embodiment, the ratio of IFN-γ:IL-4 in culture supernatants is equal to or greater than 2500. According to one embodiment, the ratio of IFN-γ:IL-4 in culture supernatants is equal to or greater than 2550. According to one embodiment, the ratio of IFN-γ:IL-4 in culture supernatants is equal to or greater than 2600. According to one embodiment, the ratio of IFN-γ:IL-4 in culture supernatants is equal to or greater than 2650. According to one embodiment, the ratio of IFN-γ:IL-4 in culture supernatants is equal to or greater than 2700. According to one embodiment, the ratio of IFN-γ:IL-4 in culture supernatants is equal to or greater than 2750. According to one embodiment, the ratio of IFN-γ:IL-4 in culture supernatants is equal to or greater than 2800. According to one embodiment, the ratio of IFN-γ:IL-4 in culture supernatants is equal to or greater than 2850. According to one embodiment, the ratio of IFN-γ:IL-4 in culture supernatants is equal to or greater than 2900. According to one embodiment, the ratio of IFN-γ:IL-4 in culture supernatants is equal to or greater than 2950. According to one embodiment, the ratio of IFN-γ:IL-4 in culture supernatants is equal to or greater than 3000.

### Cytotoxicity

The activation of CKTCs to form SCKTCs may be assessed by assaying cytotoxic activity of the CKTCs at each step of the described method.

The cytotoxic activity may be assessed by any suitable technique known to those of skill in the art. For example, a sample comprising a population of CKTCs or SCKTCs produced by the methods described herein can be assayed for cytotoxic activity after an appropriate period of time, in a standard cytotoxic assay. Such assays may include, but are not limited to, the chromium release CTL assay and the ALAMAR BLUE fluorescence assay known in the art.

According to some embodiments, a population of cells is collected by centrifugation and cytotoxicity against K562 cells (highly undifferentiated and of the granulocytic series, derived from a patient with chronic myeloid leukemia) is assessed. The K562 cell line, derived from a chronic myeloid leukemia (CML) patient and expressing B3A2 bcr-abl hybrid gene, is known to be particularly resistant to apoptotic death. (Luchetti, F. et al, Haematologica (1998) 83: 974-980). According to one embodiment, K562 target cells and SCKTCs are allocated into wells at one or more effector: target ratios, e.g. 5:1, 6:1, 7:1, 8:1, 9:1, 10:1, 11:1, 12:1, 13:1, 14:1, 15:1, 16:1, 17:1, 18:1, 19:1 or 20:1. After incubation, absorbance is detected by an enzyme-linked immunosorbent assay reader, and the killing rate can be calculated. According to other embodiments, the same assay can be carried out, where cytotoxicity against Jurkat cells (acute T leukemia) is assessed (Somanchi et al., PLoS ONE 10(10): e0141074. https://doi.org/10.1371/journal.pone.0141074).

According to some embodiments, the killing rate can be represented by the following formula:

$$Killing Rate : ( \% ) = \frac{( {OD}_{490 experimental well} - {OD}_{490 negative well} ) \times 100 \%}{( {OD}_{490 positive well} - {OD}_{490 negative well} )}$$

According to some embodiments, the killing rate of the CKTC population comprising SCKTCs ranges from about 25% to about 75%, inclusive. According to some embodiments, the killing rate of the CKTC population comprising SCKTCs ranges from about 50% to about 75%, inclusive. According to some embodiments, the killing rate of the CKTC population comprising SCKTCs is about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%.

According to some embodiments, the killing rate of the CKTC population comprising SCKTCs prepared by the described methods of the invention is increased at least 1.5-fold over control cells (e.g. cells not subject to the particular methods described in steps (c)-(e)). According to some embodiments, the killing rate of the CKTC population comprising SCKTCs prepared by the methods of the invention is increased at least 2-fold over control cells (e.g. cells not subject to the particular methods described in steps (c)-(e)). According to some embodiments, the killing rate of the CKTC population comprising SCKTCs prepared by the methods of the invention is increased at least 3-fold over control cells (e.g. cells not subject to the particular methods described in steps (c)-(e)). According to some embodiments, the killing rate of the CKTC population comprising SCKTCs prepared by the methods of the invention is increased at least 3.5-fold over control cells (e.g. cells not subject to the particular methods described in steps (c)-(e)). According to some embodiments, the killing rate of the CKTC population comprising SCKTCs prepared by the methods of the invention is increased at least 4-fold over control cells (e.g. cells not subject to the particular methods described in steps (c)-(e)). According to some embodiments, the killing rate of the CKTC population comprising SCKTCs prepared by the methods of the invention is increased at least 4.5-fold over control cells (e.g. cells not subject to the particular methods described in steps (c)-(e)). According to some embodiments, the killing rate of the CKTC population comprising SCKTCs prepared by the methods of the invention is increased at least 5-fold over control cells (e.g. cells not subject to the particular methods described in steps (c)-(e)).

### Proliferation/Expansion

The ability of the described methods of the invention to induce expansion of the SCKTCs can be evaluated by staining using the fluorescent cell staining dye carboxyfluorescein syccinimidyl ester (CFSE). To compare the initial rate of cell expansion, the cells are stained with CFSE to determine how well the various steps of the described method (i.e. steps (b)-(e)) induced the proliferation of the SCKTCs. CFSE staining provides a quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of the described method to induce proliferation of the SCKTCs is quantitated by measuring the number of cells that divided once, twice, three times and so on.

To determine how well the described method promotes long-term growth of the SCKTCs, cell growth curves can be generated. These experiments are set up as are the foregoing CFSE experiments, but no CFSE is used. Every 2-3 days of culture, cells are removed from the respective cultures and counted using a Coulter counter, which measures how many cells are present and the mean volume of the cells. The mean cell volume is the best predictor of when to restimulate the cells. In addition, the phenotypes of the cells that are expanded can be characterized to determine whether a particular subset is preferentially expanded.

Prior to each restimulation, a phenotypic analysis of the expanding cell populations is performed to determine the presence of particular markers that define the SCKTC population. According to some embodiments, prior to each restimulation, an aliquot of cells is removed from each culture and analyzed by flow cytometry, using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured. Perforin and Granzyme B intracellular staining can be used to perform a gross measure to estimate cytolytic potential.

According to some embodiments, the population of SCKTCs is expanded to from about 100- to about 1,000,000-fold, or from about 1,000- to about 1,000,000-fold, e.g., from about 1,000-fold to about 100,000-fold based on the population of starting CKTC cells, i.e., at least about 100-, at least about 200-, at least about 300-, at least about 400-, at least about 500-, at least about 600-, at least about 700-, at least about 800-, at least about 900-, at least about 1000-, at least about 2000-, at least about 3000-, at least about 4000-, at least about 5000-, at least about 6000-, at least about 7000-, at least about 8000-, at least about 9000-, at least about 10,000-, at least about 11,000-, at least about 12,000-, at least about 13,000-, at least about 14,000-, at least about 15,000-, at least about 16,000-, at least about 17,000-, at least about 18,000-, at least about 19,000-, at least about 20,000-, at least about 21,000-, at least about 22,000-, at least about 23,000-, at least about 24,000-, at least about 25,000-, at least about 26,000-, at least about 27,000-, at least about 28,000-, at least about 29,000-, at least about 30,000-, at least about 31,000-, at least about 32,000-, at least about 33,000-, at least about 34,000-, at least about 35,000-, at least about 36,000-, at least about 37,000, at least about 38,000-, at least about 39,000-, at least about 40,000-, at least about 41,000-, at least about 42,000-, at least about 43,000-, at least about 44,000-, at least about 44,000-, at least about 45,000-, at least about 46,000-, at least about 47,000-, at least about 48,000-, at least about 49,000-, at least about 50,000-, at least about 51,000-, at least about 52,000-, at least about 53,000-, at least about 54,000-, at least about 55,000-, at least about 56,000-, at least about 57,000-, at least about 58,000-, at least about 59,000-, at least about 60,000-, at least about 61,000-, at least about 62,000-, at least about 63,000-, at least about 64,000-, at least about 65,000-, at least about 66,000-, at least about 67,000-, at least about 68,000-, at least about 69,000-, at least about 70,000, at least about 71,000-, at least about 72,000-, at least about 73,000-, at least about 74,000-, at least about 75,000-, at least about 76,000-, at least about 77,000-, at least about 78,000-, at least about 79,000-, at least about 80,000-, at least about 81,000-, at least about 82,000-, at least about 83,000-, at least about 84,000-, at least about 85,000-, at least about 86,000-, at least about 87,000-, at least about 88,000-, at least about 89,000-, at least about 90,000-, at least about 91,000-, at least about 92,000-, at least about 93,000-, at least about 94,000-, at least about 95,000-, at least about 96,000-, at least about 97,000-, at least about 98,000-, at least about 99,000-, at least about 100,000-, at least about 200,000-, at least about 300,000-, at least about 400,000-, at least about 500,000-, at least about 600,000-, at least about 700,000-, at least about 800,000-, at least about 900,000-, or at least about 1,000,000-fold.

### Markers

According to some embodiments of the present invention, expansion of the SCKTCs using the methods as described herein can be determined by assessing the presence of markers that characterize the SCKTCs, and thereby determining the percent of the SCKTCs in the cell population. According to some embodiments, flow cytometry can be used to determine the presence of a subpopulation of NKT cells expressing NKT cell markers using Forward Scatter (FS) vs 90oLight Scatter bitmap the lymphocyte intact lymphocyte population. Gating (rectangular) on this bitmap, CD56 vs CD3 was measured. Gating on the double positives, Vα24 vs. Vβ11 was measured. According to some embodiments, a sub population of NKT cells can be determined by the presence of CD3 and CD56 markers (CD3+CD56+ NKT cells). According to one embodiment, binding of an anti-CD3 antibody labeled with a first fluorescent label (e.g. a commercially available fluorescently labeled anti-CD3 antibody, such as anti-CD3-pacific blue (PB) (BD Pharmingen, clone #SP34-2) and an anti-CD56 antibody labeled with a second fluorescent label (e.g. a commercially available fluorescently labeled anti-CD56 antibody, such as anti-CD56-Phycoerythrin (PE)-Cy7 (BD Pharmingen, clone #NCAM16.2)) can be used to determine expression of CD3 and CD56 in the cell population, where binding of the antibody is measured by flow cytometry for, e.g., PB fluorescence or PE fluorescence, and a gate is set based on CD3+CD56+ cells.

According to some embodiments, a subpopulation of type-I NKT cells can be determined by the presence of TCR Vα and TCR Vβ markers. According to one embodiment, binding of an anti-TCR Vα antibody labelled with a first fluorescent label (e.g. a commercially available fluorescently labeled anti-TCR Vα antibody, such as anti-TCR Vα-PE (Beckman Coulter, clone #C15)) and an anti-TCR Vβ antibody labeled with a second fluorescent label (e.g. a commercially available fluorescently labeled anti-TCR Vβ antibody, such as anti-TCR Vβ-Fluorescein isothiocyanate (FITC) (Beckman Coulter, clone #C21)) can be used to determine expression of Vα and Vβ in the cell population, where binding of the antibody is measured by flow cytometry for, e.g., PE fluorescence or FITC fluorescence, and a gate is set based on Vα+Vβ+ cells.

According to some embodiments, a subpopulation of NKT cells can be characterized by expression of the markers CD3+Vα24+. According to some embodiments, a subpopulation of type-I NKT cells is characterized by expression of the markers CD3+Vα24-. According to some embodiments, the subpopulation of type-I NKT cells includes cells characterized by the markers CD3+CD56+. According to some embodiments, the subpopulation of type-I NKT cells includes cells e characterized by expression of the markers CD3+Vα24+, CD3+Vα24-, CD3+CD56+ and mixtures thereof.

According to some embodiments, the enriched population of SCKTCs constitutes from about 40% to about 60% of the total CKTC population, i.e., about 40%, 41%, 42%, 43%, 44%, 45%, 46%, 47%, 48%, 49%, 50%, 51%, 52%, 53%, 54%, 55%, 56%, 57%, 58%, 59% or 60% of the total cell population that results from the method. Therefore, based on the number of MCs in step (c) of the method (5×10⁵/ml-3×10⁶/ml), the degree of expansion (100 to 1,000,000 fold), and the representation of SCKTCs in the total population of CTKCs (40-60%), according to some embodiments, the number of SCKTCs in the expanded, activated population enriched for SCKTCs ranges from about 2×10⁷ cells/ml to about 1.8×10¹² cells/ml.

### Methods of Use

### Subjects

The methods described herein are intended for use with any subject that may experience the benefits of these methods. Thus, “subjects,” “patients,” and “individuals” (used interchangeably) include humans as well as non-human subjects, particularly domesticated animals.

According to some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. According to some embodiments, the subject and/or animal is a human. According to some embodiments, the human is a pediatric human. According to other embodiments, the human is an adult human. According to other embodiments, the human is a geriatric human. According to other embodiments, the human may be referred to as a patient.

According to certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

According to some embodiments, the subject is a non-human animal, and therefore the disclosure pertains to veterinary use. According to some such embodiments, the non-human animal is a household pet. According to some such embodiments, the non-human animal is a livestock animal.

### Administering

The pharmaceutical compositions comprising the cell product of the present disclosure may be administered in a manner appropriate to the disease to be treated. The quantity and frequency of administration will be determined by such factors as the condition of the patient, and the type and severity of the patient's disease, although appropriate dosages may be determined by clinical trials.

The administration of the pharmaceutical compositions containing the cell product may be carried out in any manner appropriate to the particular disease, including by aerosol inhalation, injection, ingestion, transfusion, implantation or transplantation. The pharmaceutical compositions of the present disclosure may be administered to a patient parenterally, e.g., subcutaneously, intradermally, intramuscularly, by intravenous (i.v.) injection, or intraperitoneally.

According to some embodiments, the pharmaceutical compositions of the described invention also can be administered to a subject by direct injection to a desired site, or systemically. For example, the pharmaceutical compositions may be injected directly into a tumor or lymph node.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. For example, parenteral administration is contemplated to include, but is not limited to, subcutaneous, intraperitoneal, intramuscular, intrasternal injection, and kidney dialytic infusion techniques.

According to some embodiments, the pharmaceutical composition containing the population of SCKTCs can be administered to a patient daily. According to some embodiments, the pharmaceutical composition containing the population of SCKTCs can be administered to a patient by continuous infusion. According to some embodiments, the pharmaceutical composition containing the population of SCKTCs can be administered to a patient twice daily. According to some embodiments, the pharmaceutical composition containing the population of SCKTCs can be administered to a patient more than twice daily. According to some embodiments, the pharmaceutical composition containing the population of SCKTCs can be administered to a patient every other day. According to some embodiments, the pharmaceutical composition containing the population of SCKTCs can be administered to a patient twice a week. According to some embodiments, the pharmaceutical composition containing the population of SCKTCs can be administered to a patient every other week. According to some embodiments, the pharmaceutical composition containing the t population of SCKTCs can be administered to a patient every 1, 2, 3, 4, 5, or 6 months.

According to some embodiments, the pharmaceutical composition comprising a cell product containing the population of SCKTCs can be administered to a patient in a dosing regimen (dose and periodicity of administration) sufficient to maintain function of the administered SCKTCs in the bloodstream of the patient over a period of 2 weeks to a year or more, e.g., one month to one year or longer, e.g., at least 2 weeks, 4 weeks, 6 weeks, 8 weeks, 3 months, 6 months, a year, 2 years.

The frequency of the required dose will be readily apparent to the skilled artisan and will depend upon any number of factors, such as, but not limited to, the type and severity of the disease being treated, the type and age of the animal, etc.

The pharmaceutical composition comprising the cell product containing the population of SCKTCs may be co-administered with various additional therapeutic agents, e.g., cytokines, chemotherapeutic drugs, checkpoint inhibitors and/or antiviral drugs, among many others). Alternatively, the additional therapeutic agent(s) may be administered an hour, a day, a week, a month, or even more, in advance of the pharmaceutical compositions, or any permutation thereof. Further, the additional therapeutic agent(s) may be administered an hour, a day, a week, or even more, after administration of the pharmaceutical composition, or any permutation thereof. The frequency and administration regimen will be readily apparent to the skilled artisan and will depend upon any number of factors such as, but not limited to, the type and severity of the disease being treated, the age and health status of the animal, the identity of the additional therapeutic agent or agents being administered, the route of administration and the pharmaceutical composition comprising the population of SCKTCs, and the like.

According to some aspects, the present disclosure provides a method of stimulating immune cells of a subject susceptible to immune cell activation, comprising contacting an immune cell population in vivo with the pharmaceutical composition comprising a cell product containing the SCKTCs described herein, in an amount effective to stimulate the immune cell population. According to one embodiment, the immune cell population comprises a dendritic cell population. According to one embodiment, the immune cell population is a CD8+ T cell population. According to one embodiment, the immune cell population is a NK cell population. According to one embodiment, the immune cell population comprises an MHC-restricted T cell population.

According to some embodiments, the subject has a disorder susceptible to treatment comprising an immune therapy comprising administering the pharmaceutical composition containing the cell product of the present disclosure.

Exemplary embodiments include a cancer, a precancerous condition (meaning a condition that may, or is likely to) become cancer), an autoimmune disease and disorder comprising cells and/or antibodies arising from and directed against an individual's own tissues, an inflammatory disease or disorder, a tissue transplant-related disorder (meaning a disorder related to the transfer (engraftment) of human cells, tissues, or organs from a donor to a recipient with the aim of restoring function(s) in the body (transplantation), a post-transplant lymphoproliferative disorder, an allergic disorder, and an infection (meaning invasion of the body with organisms that have the potential to cause disease). For example, the pharmaceutical composition comprising the cell product containing a therapeutic amount of the population of SCKTCs of the described invention may be used to treat a condition characterized by low MHC I presentation. According to some embodiments, the pharmaceutical composition containing the SCKTC cell product may be used to treat a subject with advanced disease that cannot receive chemotherapy, e.g. the patient is unresponsive to chemotherapy or too ill to have a suitable therapeutic window for chemotherapy (e.g. a subject that is experiencing too many dose- or regimen-limiting side effects).

According to some embodiments, the term “a therapeutically effective amount” or dose does not necessarily mean an amount that is immediately therapeutically effective, but includes a dose which is capable of expansion in vivo (after administration) to provide a therapeutic effect.

Thus, there is provided a method of administering to a patient a sub-therapeutic dose that nonetheless becomes a therapeutically effective amount after expansion and activation of SCKTCs in vivo to provide the desired therapeutic effect. According to some embodiments, a sub-therapeutic dose is an amount that is less than the therapeutically effective amount.

### A Pharmaceutical Composition Comprising a Cell Product Containing an Expanded and Enriched Population of Superactivated Cytokine Killer T Cells

According to another aspect, the described invention provides a pharmaceutical composition comprising a cell product containing a therapeutic amount of an expanded and enriched population of superactivated cytokine killer T cells (SCKTCs) as an active ingredient. Such a pharmaceutical composition may contain an therapeutically effective dose of the population of SCKTCs in a form suitable for administration to a subject in addition to one or more pharmaceutically acceptable carriers. The pharmaceutical compositions of the described invention can further include one or more compatible active ingredients which are aimed at providing the composition with another pharmaceutical effect in addition to that provided by the cell product of the described invention. “Compatible” as used herein means that the active ingredients of such a composition are capable of being combined with each other in such a manner so that there is no interaction that would substantially reduce the efficacy of each active ingredient or the composition under ordinary use conditions.

According to some embodiments, the expanded and enriched superactivated population of cytokine killer T cells (SCKTCs) is characterized by one or more of a modulation of secretion of a cytokine, stimulated proliferation of the population of SCKTCs, modulated expression of one or more markers on the cell surface of the SCKTCs or increased cytotoxic activity by the SCKTCs against a target cell population.

### Cytokine Secretion

According to some embodiments, the expanded and enriched population of SCKTCs is characterized by modulation of expression of one or more cytokine selected from the group consisting of IL-4, IL-5, IL-6, or IL-10 or IFNγ. According to some embodiments, the expanded and enriched population of SCKTC cells comprises cells with a profile of expression of cytokines comprising low expression of one or more cytokines selected from the group consisting of IL-4, IL-5, IL-6, and IL-10, and high expression of IFNγ. According to some embodiments, cytokine production by the enriched population of SCKTCs is characterized as IL-5-, IL-6-, IL-, IFN-4 low, and IFNγ high.

According to some embodiments, the amount of IFN-γ produced by the expanded and enriched population of SCKTCs is about 5000 pg/ml or greater.

According to some embodiments, the amount of IL-4 produced by the expanded and enriched population of SCKTCs is about 5 pg/ml. According to one embodiment, the amount of IL-4 produced by the expanded and enriched population of SCKTCs is about 4.5 pg/ml. According to one embodiment, the amount of IL-4 produced by the expanded and enriched population of SCKTCs is about 4 pg/ml. According to one embodiment, the amount of IL-4 produced by the expanded and enriched population of SCKTCs is about 3.5 pg/ml. According to one embodiment, the amount of IL-4 produced by the expanded and enriched population of SCKTCs is about 3 pg/ml. According to one embodiment, the amount of IL-4 produced by the expanded and enriched population of SCKTCs is about 2.5 pg/ml. According to one embodiment, the amount of IL-4 produced by the expanded and enriched population of SCKTCs is about 2 pg/ml. According to one embodiment, the amount of IL-4 produced by the expanded and enriched population of SCKTCs is about 1.5 pg/ml. According to one embodiment, the amount of IL-4 produced by the expanded and enriched population of SCKTCs is about 1 pg/ml. According to one embodiment, the amount of IL-4 produced by the expanded and enriched population of SCKTCs is between about 1.0 and about 5 pg/ml. According to one embodiment, the amount of IL-4 produced by the expanded and enriched population of SCKTCs is between about 1.5 and about 5 pg/ml. According to one embodiment, the amount of IL-4 produced by the expanded and enriched population of SCKTCs is between about 2.0 and about 5 pg/ml. According to one embodiment, the amount of IL-4 produced by the expanded and enriched population of SCKTCs is between about 2.5 and about 5 pg/ml. According to one embodiment, the amount of IL-4 produced by the expanded and enriched population of SCKTCs is between about 3.0 and about 5 pg/ml. According to one embodiment, the amount of IL-4 produced by the expanded and enriched population of SCKTCs is between about 3.5 and about 5 pg/ml. According to one embodiment, the amount of IL-4 produced by the expanded and enriched population of SCKTCs is between about 4.0 and about 5 pg/ml. According to one embodiment, the amount of IL-4 produced by the expanded and enriched population of SCKTCs is between about 4.5 and about 5 pg/ml.

According to some embodiments, the ratio of IFNγ to IL-4 is an indicator of one or more T cell effector functions (such as cell killing and cell activation), of the control population of CKTCs and the expanded and enriched population of SCKTCs. According to one embodiment, the ratio of IFN-γ:IL-4 in culture supernatants is equal to or greater than 1000. According to one embodiment, the ratio of IFN-γ:IL-4 in culture supernatants is equal to or greater than 1200. According to one embodiment, the ratio of IFN-γ:IL-4 in culture supernatants is equal to or greater than 1300. According to one embodiment, the ratio of IFN-γ:IL-4 in culture supernatants is equal to or greater than 1400. According to one embodiment, the ratio in culture supernatants of IFN-γ:IL-4 is equal to or greater than 1500. According to one embodiment, the ratio of IFN-γ:IL-4 in culture supernatants is equal to or greater than 1550. According to one embodiment, the ratio of IFN-γ:IL-4 in culture supernatants is equal to or greater than 1600. According to one embodiment, the ratio of IFN-γ:IL-4 in culture supernatants is greater than 1650. According to one embodiment, the ratio of IFN-γ:IL-4 in culture supernatants is equal to or greater than 1700. According to one embodiment, the ratio of IFN-γ:IL-4 in culture supernatants is equal to or greater than 1750. According to one embodiment, the ratio of IFN-γ:IL-4 in culture supernatants is equal to or greater than 1800. According to one embodiment, the ratio of IFN-γ:IL-4 in culture supernatants is equal to or greater than 1850. According to one embodiment, the ratio of IFN-γ:IL-4 in culture supernatants is equal to or greater than 1900. According to one embodiment, the ratio of IFN-γ:IL-4 in culture supernatants is greater than 1950. According to one embodiment, the ratio of IFN-γ:IL-4 in culture supernatants is equal to or greater than 2000. According to one embodiment, the ratio of IFN-γ:IL-4 is equal to or greater than 2050. According to one embodiment, the ratio of IFN-γ:IL-4 in culture supernatants is equal to or greater than 2100. According to one embodiment, the ratio of IFN-γ:IL-4 in culture supernatants is equal to or greater than 2150. According to one embodiment, the ratio of IFN-γ:IL-4 in culture supernatants is equal to or greater than 2200. According to one embodiment, the ratio of IFN-γ:IL-4 in culture supernatants is equal to or greater than 2250. According to one embodiment, the ratio of IFN-γ:IL-4 in culture supernatants is equal to or greater than 2300. According to one embodiment, the ratio of IFN-γ:IL-4 in culture supernatants is equal to or greater than 2350. According to one embodiment, the ratio of IFN-γ:IL-4 in culture supernatants is equal to or greater than 2400. According to one embodiment, the ratio of IFN-γ:IL-4 in culture supernatants is equal to or greater than 2450. According to one embodiment, the ratio of IFN-γ:IL-4 in culture supernatants is equal to or greater than 2500. According to one embodiment, the ratio of IFN-γ:IL-4 in culture supernatants is equal to or greater than 2550. According to one embodiment, the ratio of IFN-γ:IL-4 in culture supernatants is equal to or greater than 2600. According to one embodiment, the ratio of IFN-γ:IL-4 in culture supernatants is equal to or greater than 2650. According to one embodiment, the ratio of IFN-γ:IL-4 in culture supernatants is equal to or greater than 2700. According to one embodiment, the ratio of IFN-γ:IL-4 in culture supernatants is equal to or greater than 2750. According to one embodiment, the ratio of IFN-γ:IL-4 in culture supernatants is equal to or greater than 2800. According to one embodiment, the ratio of IFN-γ:IL-4 in culture supernatants is equal to or greater than 2850. According to one embodiment, the ratio of IFN-γ:IL-4 in culture supernatants is equal to or greater than 2900. According to one embodiment, the ratio of IFN-γ:IL-4 in culture supernatants is equal to or greater than 2950. According to one embodiment, the ratio of IFN-γ:IL-4 in culture supernatants is equal to or greater than 3000.

Enriched Population of SCTKCs

The ability of the described methods of the invention to induce expansion of the expanded and enriched population of SCKTCs can be evaluated by staining using the fluorescent cell staining dye carboxyfluorescein syccinimidyl ester (CFSE). To compare the initial rate of cell expansion, CKTCs are stained with CFSE to determine how well the various steps of the described method (i.e. steps (c)-(e)) induced the proliferation of the SCKTCs. CFSE staining provides a quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of the described method to induce proliferation of the SCKTCs is quantitated by measuring the number of cells that divided once, twice, three times and so on.

To determine how well the described method promotes long-term growth of the SCKTCs, cell growth curves can be generated. These experiments are set up as are the foregoing CFSE experiments, but no CFSE is used. Every 2-3 days of culture, cells are removed from the respective cultures and counted using a Coulter counter, which measures how many cells are present and the mean volume of the cells. The mean cell volume is the best predictor of when to restimulate the cells. In addition, the phenotypes of the cells that are expanded can be characterized to determine whether a particular subset is preferentially expanded.

Prior to each restimulation, a phenotypic analysis of the expanding cell populations is performed to determine the presence of particular markers that define the SCKTC population. According to some embodiments, prior to each restimulation, an aliquot of cells is removed from each culture and analyzed by flow cytometry, using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured. Perforin and Granzyme B intracellular staining can be used to perform a gross measure to estimate cytolytic potential.

According to some embodiments, the population of SCKTCs is expanded to from about 100 to about 1,000,000 fold, or from about 1,000 to about 1,000,000 fold, e.g., from about 1,000 fold to about 100,000 fold based on the population of starting CKTC cells, i.e., at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10,000, at least about 11,000, at least about 12,000, at least about 13,000, at least about 14,000, at least about 15,000, at least about 16,000, at least about 17,000, at least about 18,000, at least about 19,000, at least about 20,000, at least about 21,000, at least about 22,000, at least about 23,000, at least about 24,000, at least about 25,000, at least about 26,000, 27,000, at least about 28,000, 29,000, 30,000, at least about 31,000, at least about 32,000, at least about 33,000, at least about 34,000, at least about at least about 35,000, at least about 36,000, at least about 37,000, at least about 38,000, at least about 39,000, at least about 40,000, at least about 41,000, at least about 42,000, at least about 43,000, at least about 44,000, at least about 44,000, at least about 45,000, at least about 46,000, at least about 47,000, at least about 48,000, at least about 49,000, 5 at least about 0,000, at least about 51,000, at least about 52,000, at least about 53,000, at least about 54,000, at least about 55,000, at least about 56,000, at least about 57,000, at least about 58,000, at least about 59,000, at least about 60,000, at least about 61,000, at least about 62,000, at least about 63,000, at least about 64,000, at least about 65,000, at least about 66,000, at least about 67,000, at least about 68,000, at least about 69,000, at least about 70,000, at least about 71,000, at least about 72,000, at least about 73,000, at least about 74,000, at least about 75,000, 7 at least about 76,000, at least about 77,000, at least about 78,000, at least about 79,000, at least about 80,000, at least about 81,000, at least about 82,000, at least about 83,000, at least about 84,000, at least about 85,000, at least about 86,000, at least about 87,000, at least about 88,000, at least about 89,000, at least about 90,000, at least about 91,000, at least about 92,000, at least about 93,000, at least about 94,000, at least about 95,000, at least about 96,000, at least about 97,000, at least about 98,000, at least about 99,000, at least about 100,000, at least about 200,000, at least about 300,000, at least about 400,000, at least about 500,000, at least about 600,000, at least about 700,000, at least about 800,000, at least about 900,000, or at least about 1,000,000 fold.

With regard to stimulated proliferation, according to some embodiments, the expanded and enriched population of SCKTCs constitutes from about 40% to about 60% of the total CTKC cell population, i.e., about 40%, 41%, 42%, 43%, 44%, 45%, 46%, 47%, 48%, 49%, 50%, 51%, 52%, 53%, 54%, 55%, 56%, 57%, 58%, 59% or 60% of the total CTKC cell population.

According to some embodiments, the number of SCKTCs in the expanded population enriched for SCKTCs ranges from about 2×10⁷ cells/ml to about 1.8×10¹² cells/ml.

Marker Expression

According to some embodiments, flow cytometry (for example, using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured.) can be used to characterize expression of cell markers by the expanded and enriched population of SCKTCs. According to some embodiments, the expanded and enriched population of SCKTCs comprises a subpopulation of cells expressing NKT cell markers. According to some such embodiments, the subpopulation of cells expressing NKT markers can be determined by the presence of CD3 and CD56 markers. According to some embodiments, binding of an anti-CD3 antibody labeled with a first fluorescent label (e.g. a commercially available fluorescently labeled anti-CD3 antibody, such as anti-CD3-pacific blue (PB) (BD Pharmingen, clone #SP34-2))) and an anti-CD56 antibody labeled with a second fluorescent label (e.g. a commercially available fluorescently labeled anti-CD56 antibody, such as anti-CD56-Phycoerythrin (PE)-Cy7 (BD Pharmingen, clone #NCAM16.2)) can be used to determine expression of CD3 and CD56 in the expended and enriched SCKTC population, where binding of the antibody is measured by flow cytometry for, e.g., PB fluorescence or PE fluorescence, and a gate is set based on CD3+CD56+ cells.

According to some embodiments, the expanded and enriched population of SCKTCs comprises a subpopulation of cells expressing type-I NKT markers. According to some such embodiments, the subpopulation of cells expressing type 1 NKT markers can be determined by the presence of TCR Vα and TCR Vβ markers. According to some embodiments, binding of an anti-TCR Vα antibody labelled with a first fluorescent label (e.g. a commercially available fluorescently labeled anti-TCR Vα antibody, such as anti-TCR Vα-PE (Beckman Coulter, clone # C15)) and an anti-TCR Vβ antibody labeled with a second fluorescent label (e.g. a commercially available fluorescently labeled anti-TCR Vβ antibody, such as anti-TCR Vβ-Fluorescein isothiocyanate (FITC) (Beckman Coulter, clone #C21)) can be used to determine expression of Vα and Vβ in the cell population, where binding of the antibody is measured by flow cytometry for, e.g., PE fluorescence or FITC fluorescence, and a gate is set based on Vα+Vβ+ cells.

According to some embodiments, the subpopulation of cells expressing type-I NKT cell markers can comprise cells characterized by expression of the markers CD3+Vα24+. According to some embodiments, the subpopulation of cells expressing type 1 NKT cells markers comprises cells characterized by expression of the markers CD3+Vα24-. According to some embodiments, the subpopulation of cells expressing type 1 NKT cells markers includes cells that are characterized by the markers CD3+CD56+. According to some embodiments, the subpopulation of cells expressing type 1 NKT cells markers includes cells that are characterized by expression of the markers CD3+Vα24+, CD3+Vα24-, CD3+CD56+ and mixtures thereof.

### Cytotoxic Activity

Cytotoxic activity may be assessed by any suitable technique known to those of skill in the art. For example, the pharmaceutical composition comprising a cell product containing an expanded and enriched population of SCKTCs as described herein can be assayed for cytotoxic activity at an appropriate period of time in a standard cytotoxic assay. Such assays may include, but are not limited to, the chromium release CTL assay and the ALAMAR BLUE fluorescence assay known in the art.

According to some embodiments, a sample of a population of effector T cells is collected by centrifugation and its cytotoxicity assessed against target K562 cells (highly undifferentiated and of the granulocytic series, derived from a patient with chronic myeloid leukemia). The K562 cell line, derived from a chronic myeloid leukemia (CML) patient and expressing B3A2 bcr-abl hybrid gene, is known to be particularly resistant to apoptotic death. (Luchetti, F. et al, Haematologica (1998) 83: 974-980). According to one embodiment, replicate samples of K562 target cells and effector SCKTCs prepared according to the described methods of the invention are allocated into wells at one or more effector: target ratios, e.g. 5:1, 6:1, 7:1, 8:1, 9:1, 10:1, 11:1, 12:1, 13:1, 14:1, 15:1, 16:1, 17:1, 18:1, 19:1 or 20:1. After incubation, absorbance is detected by an enzyme-linked immunosorbent assay reader, and the killing rate can be calculated. According to other embodiments, the same assay can be carried out, where cytotoxicity against Jurkat cells (acute T leukemia) is assessed (Somanchi et al., PLoS ONE 10(10): e0141074. https://doi.org/10.1371/journal.pone.0141074).

According to some embodiments, the killing rate can be represented by the following formula:

$$Killing Rate : ( \% ) = \frac{( {OD}_{490 experimental well} - {OD}_{490 negative well} )}{( {OD}_{490 positive well} - {OD}_{490 negative well} )} \times 100 \% .$$

According to some embodiments, the killing rate of the expanded and enriched population of SCKTCs ranges from about 25% to about and 75%, inclusive. According to some embodiments, the killing rate of the expanded and enriched population of SCKTCs ranges from about 50% to about and 75%, inclusive. According to some embodiments, the killing rate of the expanded and enriched population of SCKTCs is about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%.

According to some embodiments, the killing rate of the expanded and enriched population of SCKTCs prepared by the described methods of the invention is increased at least 1.5 fold over control CKTCs (e.g. cells not subject to the particular methods described in steps (c)-(e)). According to some embodiments, the killing rate of the expanded and enriched population of SCKTCs prepared by the methods of the invention is increased at least 2 fold over control CTKCs (e.g. cells not subject to the particular methods described in steps (c)-(e)). According to some embodiments, the killing rate of the expanded and enriched population of SCKTCs prepared by the methods of the invention is increased at least 3 fold over control CTKCs (e.g. cells not subject to the particular methods described in steps (c)-(e)). According to some embodiments, the killing rate of the expanded and enriched population of SCKTCs prepared by the methods of the invention is increased at least 3.5 fold over control CKTCs (e.g. cells not subject to the particular methods described in steps (c)-(e)). According to some embodiments, the killing rate of the expanded and enriched population of SCKTCs prepared by the methods of the invention is increased at least 4 fold over control CKTCs (e.g. cells not subject to the particular methods described in steps (c)-(e)). According to some embodiments, the killing rate of the expanded and enriched population of SCKTCs prepared by the methods of the invention is increased at least 4.5 fold over control CKTCs (e.g. cells not subject to the particular methods described in steps (c)-(e)). According to some embodiments t, the killing rate of the expanded and enriched population of SCKTCs prepared by the methods of the invention is increased at least 5 fold over control CKTCs (e.g. cells not subject to the particular methods described in steps (c)-(e)).

According to some embodiments, the expanded and enriched population of SCKTCs are characterized by an IFN gamma:IL4 ratio of at least 1000, a killing rate increased at least 1.5 fold over control cells, or both.

Formulations of the pharmaceutical composition suitable for parenteral administration comprise the active ingredient combined with a pharmaceutically acceptable carrier, such as sterile water or sterile isotonic saline. Exemplary carrier solutions also can contain buffers, diluents and other suitable additives. The term “buffer” as used herein refers to a solution or liquid whose chemical makeup neutralizes acids or bases without a significant change in pH. Examples of buffers envisioned by the described invention include, without limitation, Dulbecco's phosphate buffered saline (PBS), Ringer's solution, 5% dextrose in water (D5W), normal/physiologic saline (0.9% NaCl). In some embodiments, the infusion solution is isotonic to subject tissues.

Exemplary pharmaceutical compositions of the described invention may comprise a suspension or dispersion of cells in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A dispersion is a two-phase system, in which one phase (e.g., particles) is distributed in a second or continuous phase. A suspension is a dispersion in which a finely-divided species is combined with another species, with the former being so finely divided and mixed that it does not rapidly settle out. Among the acceptable vehicles and solvents that may be employed are water, Ringer's solution, and isotonic sodium chloride (saline) solution.

Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

Formulations of the pharmaceutical composition may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the disclosure may be prepared/formulated, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

According to some embodiments, the pharmaceutical compositions of the described invention may be administered initially, and thereafter maintained by further administrations. For example, according to some embodiments, the pharmaceutical compositions of the described invention may be administered by one method of injection, and thereafter further administered by the same or by different method.

The pharmaceutical composition of the disclosure may be prepared, packaged, or sold in bulk, as a single unit dose, or as a plurality of single unit doses. As used herein, a “unit dose” is a discrete amount of the pharmaceutical composition comprising the cell product comprising a predetermined amount of the active ingredient, i.e., the expanded and enriched population of SCKTCs. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, according to some embodiments, a pharmaceutical composition of the disclosure may further comprise one or more additional pharmaceutically active agents, e.g., cytokines, chemotherapeutic drugs, checkpoint inhibitors and/or antiviral drugs, among many others.

According to some embodiments, a protein stabilizing agent can be added to the cell product comprising the expended and enriched population of SCKTCs after manufacturing, for example albumin, which may act as a stabilizing agent. According to some embodiments, the albumin is human albumin. According to some embodiments, the albumin is recombinant human albumin. According to some embodiments, the minimum amounts of albumin employed in the formulation may be about 0.5% to about 25% w/w, i.e., about 0.5%, about 1.0%, about 2.0, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25% w/w, including intermediate values, such as about 12.5% w/w.

According to some embodiments, the pharmaceutical composition comprises a stabilizing amount of serum. The term “stabilizing amount” as used herein refers to the amount of serum that, when included in the formulation of the pharmaceutical composition of the described invention comprising enriched SCKTCs, enables these cells to retain their T cell effector activity. According to some embodiments, the serum is human serum autologous to a human patient. According to some embodiments, the serum is synthetic serum. According to some embodiments the stabilizing amount of serum is at least about 10% (v/v).

According to some embodiments, the methods of the present invention comprise the further step of preparing the pharmaceutical composition by adding a pharmaceutically acceptable excipient, in particular an excipient as described herein, for example a diluent, stabilizer and/or preservative.

The term “excipient” as employed herein is a generic term to cover all ingredients added to the SCKTC population that do not have a biological or physiological function, which are nontoxic and do not interact with other components.

Once the final formulation of the pharmaceutical composition has been prepared it will be filled into a suitable container, for example an infusion bag or cryovial.

According to some embodiments, the methods according to the present disclosure comprises the further step of filling the pharmaceutical composition comprising the cell product containing the expanded and enriched population of SCKTCs or a pharmaceutical formulation thereof into a suitable container, such as an infusion bag and sealing the same to form the cell product.

According to some embodiments, the product comprising the container filled with the pharmaceutical composition comprising the cell product comprising the expanded and enriched population of SCKTCs of the present disclosure is frozen for storage and transport, for example at about −135° C., for example in the vapor phase of liquid nitrogen. According to some such embodiments, the formulation may also contain a cryopreservative, such as DMSO. The quantity of DMSO is generally about 20% or less, such as about 10%, 11%, 12%, 13%, 14%, 15%, 16%, 17%, 18%, 19%, or 20% v/v.

According to some embodiments, the process of the present disclosure comprises the further step of freezing the pharmaceutical composition, or the cell product comprising the expanded and enriched population of SCKTCs of the present disclosure. According to one embodiment, freezing occurs by a controlled rate freezing process, for example reducing the temperature by 1° C. per minute to ensure the crystals formed are small and do not disrupt cell structure. This process may be continued until the sample has reached about −100° C.

Controlled- or sustained-release formulations of the pharmaceutical composition of the disclosure may be made by adapting otherwise conventional technology. The term “controlled release” as used herein is intended to refer to any drug-containing formulation in which the manner and profile of drug release from the formulation are controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant levels of a drug over an extended time period. The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.” The term “long-term” release, as used herein, means that the drug formulation is constructed and arranged to deliver therapeutic levels of the active ingredient over a prolonged period of time, e.g., days.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations may include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. For parenteral application, suitable vehicles consist of solutions, e.g., oily or aqueous solutions, as well as suspensions, emulsions, or implants. Aqueous suspensions may contain substances, which increase the viscosity of the suspension and include, for example, sodium carboxymethyl cellulose, sorbitol and/or dextran.

According to some embodiments, the present disclosure provides a method of transporting a cell product comprising the expanded and enriched population of SCKTCs according to the present disclosure from the place of manufacture, or a convenient collection point, to a therapeutic facility. According to some embodiments, the temperature of the cell product is maintained during such transporting. According to some embodiments, for example, the pharmaceutical composition can be stored below 0° C., such as −135° C. during transit. According to some embodiments, temperature fluctuations of the pharmaceutical composition are monitored during storage and/or transport.

The publications discussed herein are provided solely for their disclosure prior to the filing date of the present application and each is incorporated by reference in its entirety. Nothing herein is to be construed as an admission that the described invention is not entitled to antedate such publication by virtue of prior invention. Further, the dates of publication provided may be different from the actual publication dates which may need to be independently confirmed.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Isolation of Mononuclear Cells (MCs) from Peripheral Blood

The following procedure describes isolation of MC from blood, more specifically peripheral blood, from a human subject:

1. 30 ml-50 ml of heparin anticoagulated human peripheral blood was obtained and placed in a centrifuge tube. The peripheral blood was diluted with saline in a proportion of 1:1, and mixed until uniform.

2. A new 50 mL centrifuge tube was filled with 15 ml of lymphocyte separation solution (Ficoll-Paque); the uniformly diluted blood is then slowly layered onto the lymphocyte separation solution by adding it along the tube wall in a ficoll: diluting blood volume ratio of 1:2, forming a clear stratification therebetween, and the mixture was centrifuged at 3000 rpm for 30 min.

3. After completion of the centrifugation, mononuclear cells at the interface between the plasma and the Ficoll-Paque layer were collected, placed into a new 50 ml centrifuge tube, rinsed with 30 ml of X-VIVO-15 medium, and centrifuged at 800 g for 5 min. The supernatant was then removed.

4. The mixture was added to 20 ml of X-VIVO-15 medium, mixed uniformly by pipetting and centrifuged at 200 g under room temperature for 10 min, and then the supernatant was removed. Cells were resuspended in 10 ml of X-VIVO-15 medium and counted.

### Example 2. Induction of Differentiation of Peripheral Blood Mononuclear Cells (PBMCs) into Dendritic Cells (DC)

The following procedure describes a process for the induction of differentiation of PBMCs into dendritic cells.

1. The concentration of PBMCs was adjusted to 1×10⁶ cells/ml with RPMI 1640 medium containing 10% FBS, and the cells were plated in a T25 culture flask for static culturing in 5% of CO₂ at 37° C. for 1 h.

2. The supernatant containing non-adhered cells, was removed from the culture flask. The cells that remained were rinsed with RPMI 1640 medium containing 10% FBS twice, then transferred into 5 ml of RPMI 1640 medium containing 10% FBS, supplemented with cytokines GM-CSF and IL-4, at concentrations of 500 U/ml and 50 ng/ml, respectively.

3. At day 4, the culture system was supplemented with 3 ml of medium containing GM-CSF and IL-4 with said working concentration (50 ng/ml).

4. At day 6, alpha-GalCer was added to the culture system until a working concentration of 100 ng/ml was met. This step was performed to load the dendritic cells with alpha-GalCer.

5. At day 7, dendritic cells loaded with alpha-GalCer were collected.

### Example 3. In Vitro Amplification (Meaning Expansion) of Cytokine Killer T Cells (CKTCs) with High Killing Activity

The following procedure describes a process for the in vitro amplification of cytokine killer T cells (CKTCs) to form superactivated CKTCs that have high killing activity.

1. The concentration of PBMCs was adjusted to 3×10⁶ cells/ml with X-VIVO-15 medium. Alpha-GalCer was added to the culture system until a working concentration of 100 ng/ml was met, and the cells were plated in a 6-well plate.

2. At day 3, the culture medium in the culture system was changed and alpha-GalCer was added until the working concentration of 100 ng/ml was met.

3. At day 7, the dendritic cells loaded with alpha-GalCer (about 1×10⁵ cells) obtained in Example 2 were added into a culture system comprising a population of CKTCs, and the following stimulating factors were added at working concentrations as follows: 100 ng/ml alpha-GalCer, 100 U/ml of IL-2 and 20 ng/ml of IL-7. A tube of PBMC was recovered to induce their differentiation into dendritic cells for secondary stimulation of CKTCs in the same manner as described in Example 2.

4. At day 10, the media in the culture system was replenished, and alpha-GalCer, IL-2 and IL-7 were added until the respective working concentrations were met (100 ng/ml alpha-GalCer, 100 U/ml of IL-2 and 20 ng/ml of IL-7).

5. At day 14, the dendritic cells loaded with alpha-GalCer were again added into the CKTC cell culture system, stimulating factors alpha-GalCer, IL-2 and IL-7 were supplemented to respective working concentrations, and IL-15 was added into the culture system up to 20 ng/ml.

6. At day 17, the culture medium in the culture system was replenished and alpha-GalCer, IL-2, IL-7 and IL-15 were added until respective working concentrations were met (100 ng/ml alpha-GalCer, 100 U/ml of IL-2 and 20 ng/ml of IL-7).

7. At day 20, the culture medium in the culture system was replenished and alpha-GalCer, IL-2, IL-7 and IL-15 were added until the respective working concentrations were met (100 ng/ml alpha-GalCer, 100 U/ml of IL-2 and 20 ng/ml of IL-7), and IL-12 was added until a working concentration of 20 ng/ml was met.

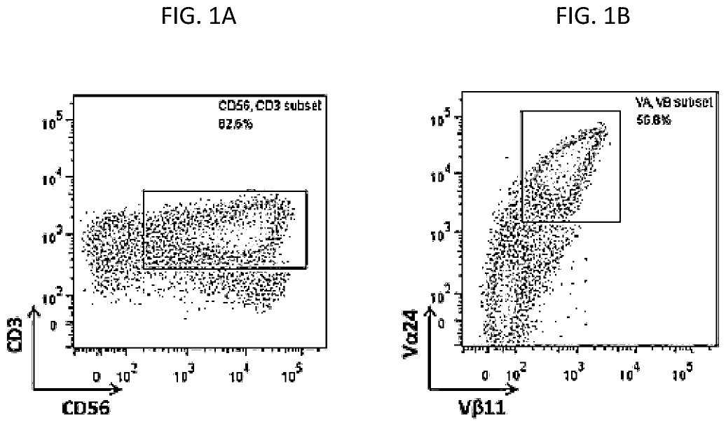

8. At day 21, cells were collected. 100 μl of the expanded superactivated CTKC cell product were removed and transferred into the following fluorescent antibody: anti-TCR Vα-PE (Beckman Coulter, clone #C15), anti-TCR Vβ-FITC (Beckman Coulter, clone #C21), anti-CD3-PB (BD Pharmingen, clone #SP34-2), anti-CD56-PE-Cy7 (BD Pharmingen, clone #NCAM16.2), After incubation at 4° C. for 30 min, the proportion of target expanded superactivated CKTCs expressing type 1-NKT cell markers was measured by flow cytometry using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured. As shown in FIG. 1, the expanded superactivated CKTC product comprises a population of cells expressing NKT markers CD3+CD56+, with up to 56.8% of the cells expressing type 1-NKT markers.

In summary, about 90% of the population of SCKTCs comprises CD3+ T cells, and about 50% of the population of SCKTCs comprises type 1-NKT cells (data not shown).

### Example 4. Effects of Time of Adding Cytokines IL-2 and IL-7 on Amplification/Expansion of CKTCs

Under the same culture conditions (37° C., CO₂ concentration of 5%), the effect of IL-2, IL-7 or both IL-2 and IL-7 on CKTCs cultured by different processes A, B, C and D was tested, where alpha-GalCer was added at the beginning of culture and maintained until the completion of culture. For Group A, IL-2 was added simultaneously at the beginning of culture; for Group B, IL-2 and IL-7 were added simultaneously at the beginning of culture; for Group C, IL-2 and IL-7 were added at day 3; and for Group D, IL-2 and IL-7 were added at day 7.

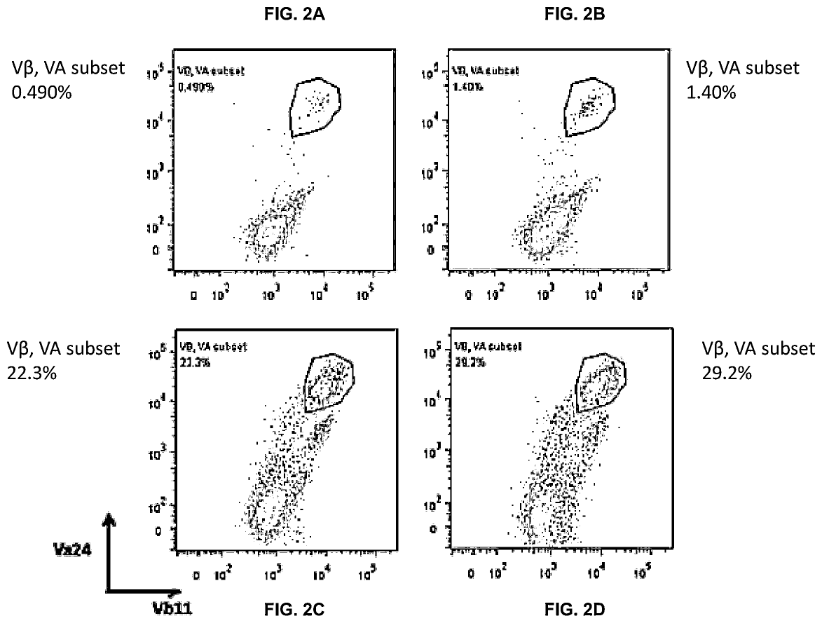

On day 21, 100 μl of the CKTCs expanded by the processes A, B, C and D were removed and incubated with the following fluorescent antibodies, respectively: TCR Vα-PE and TCR Vβ-FITC. After incubation at 4° C. for 30 min, the proportion of target cells was measured by flow cytometry using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured. As shown in FIG. 2, the proportion of CKTC cells expressing type-I NKT cell markers in the CKTCs of Groups A to D gradually increased. The results showed that the addition of cytokines IL-2 and IL-7 at day 7 can lead to preferential expansion of the CKTC cells expressing type-I NKT cell markers, and significantly improved the purity of this cell population in the expanded population of CKTC cells.

### Example 5. Effects of Time of Adding Cytokine IL-15 on the Proportion of Expanded CKTCs

Under the same culture conditions (37° C., CO₂ concentration of 5%), the effect of IL-15 on CKTCs cultured by different processes A, B, C and D was tested, where alpha-GalCer was added at the beginning of culture, and IL-2 and IL-7 added at day 7, until completion of culture. For Group A, no IL-15 was added; for Group B, IL-15 was added simultaneously at the beginning of culture; for Group C, IL-15 was added at day 7; and for Group D, IL-15 was added at day 14.

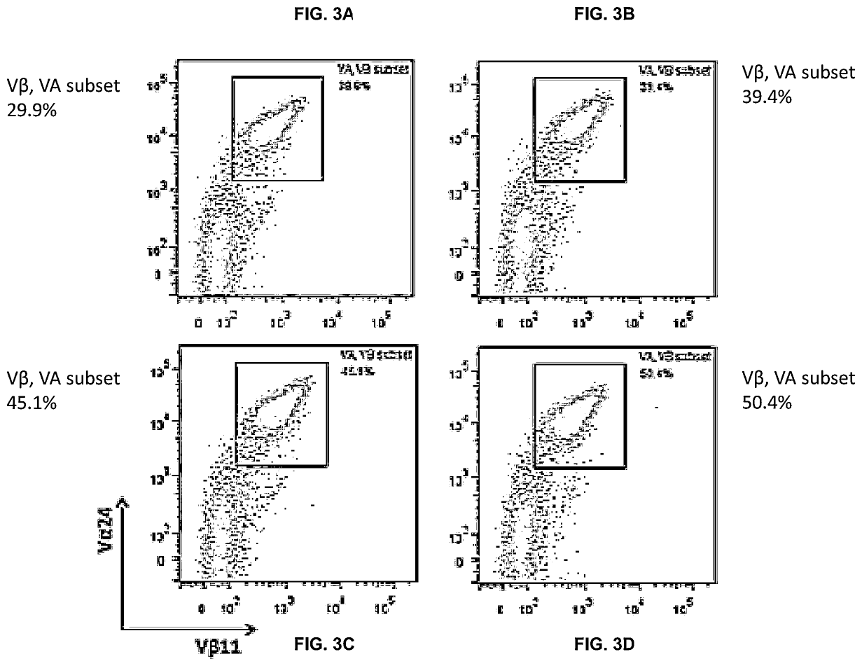

On day 21, 100 μl of the CKTC population expanded by the processes A, B, C and D was removed and incubated with the following fluorescent antibody respectively: anti-TCR Vα-PE, anti-TCR Vβ-FITC, anti-CD3-PB and anti-CD56-PE-Cy7. After incubation at 4° C. for 30 min, the proportion of CKTC cells expressing type 1 NKT cell markers in each group was measured by flow cytometry using Forward Scatter (FS) vs 90° Light Scatter bitmap the lymphocyte intact lymphocyte population. Gating (rectangular) on this bitmap, CD56 vs CD3 was measured. Gating on the double positives, Vα24 vs. Vβ11 was measured. As shown in FIG. 3, the proportion of cells expressing type-I NKT cell markers in the CKTC population of Group D is superior to the proportion of cells expressing type 1-NKT cell markers in the other three groups.

### Example 6. Effect of Time of Adding Cytokine IL-15 on the Ability of Amplified/Expanded Populations of CTKC Cells to Secrete Cytokines

The ratio of IFN-γ to IL-4 in the supernatant of the expanded CTKC cell population was used as an indicator for evaluating the effector function of the expanded CTKC cell population.

Using CBA (Cytometric Bead Array), the ratio of IFN-γ:IL-4 in each of the four groups of culture supernatant in Example 5 were measured, by which the ability of the expanded CKTCs expressing NKT markers to secrete effector cytokines was evaluated. The results are shown in Table 2.

TABLE 2
Effect of time of adding cytokines IL-15 on the ability of amplified
CKTC populations expressing NKT markers to secrete cytokines

| | Group A | Group B | Group C | Group D |
|---|---|---|---|---|
| IFN-γ (pg/ml) | >5000 | >5000 | >5000 | >5000 |
| IL-4 (pg/ml) | 4.47 | 3.21 | 3.07 | 1.84 |
| IFN-γ: IL-4 | >1118 | >1558 | >1629 | >2717 |

The results show that addition of IL-15 at day 14 in culture (Group D) significantly increased the ratio of IFN-γ:IL-4 in the supernatant of the expanded population of CTKCs so that the ability of the expanded population of CTKCs to secrete effector cytokines is improved, compared to controls.

### Example 7. Effect of Time of Adding Cytokine IL-15 on the Killing Ability of the Expanded Population of CTKCs

Lactate dehydrogenase (LDH) is a stable cytoplasmic enzyme, which may be released into the extracellular milieu upon lysis of a cell and catalyze a tetrazolium salt (INT) on its substrate to produce red products, the amount of which is in proportion to that of the cell lysates. In this example, by measuring the amount of INT in the killing system, the activity of the expanded population of CTKCs to kill target cells was evaluated. An LDH kit (#CK12, DOJINDO) was used for measurement, in accordance with the instructions provided by the producer or supplier.

K562 target cells were obtained and centrifuged, and the density of the target cells was adjusted to 1×10⁵ cells/mL. The expanded and activated population of CTKC effector cells cultured in processes A and D above were collected by centrifugation, and Effector:Target ratios adjusted to 5:1, 10:1 and 20:1. For each group, duplicate wells were provided. Following incubation in 5% of CO₂ at 37° C. for 4 h and sufficient dissolution of precipitates, absorbance was detected by an enzyme-linked immunosorbent assay reader, and the killing rate was calculated. The killing rate is determined using the following formula: Killing Rate (%)=(OD490experimental well−OD490negative well)/(OD490positive well−OD490negative well)×100%. The results are shown in Table 3.

TABLE 3
Effect of cytokines IL-15 on the killing ability
of expanded, activated CKTCs

| | Effector:Target ratio | | |
|---|---|---|---|
| | 5:1 | 10:1 | 20:1 |
| Group A | 12.41 | 24.1 | 37.09 |
| Group D | 32.341 | 46.7 | 58.75 |

The results showed that addition of IL-15 at day 14 of culture (Group D) significantly improved the killing ability of the expanded and activated population of CKTCs.

### Example 8. Effect of Time of Adding Cytokine IL-12 on the Proportion of CKTCs Expressing Type-I NKT Cell Markers in the Expanded Population of CKTCs

Under the same culture conditions (37° C., CO₂ concentration of 5%), the effector function of CKTCs cultured by the different processes of Groups A, B, C and D were tested, where alpha-GalCer was added at the beginning of culture, IL-2 and IL-7 were added at day 7, and IL-15 was added at day 14, until the completion of culture. For Group A, no IL-12 was added; for Group B, IL-12 was added simultaneously at the beginning of culture; for Group C, IL-12 was added at day 7; and for Group D, IL-12 was added at day 20.

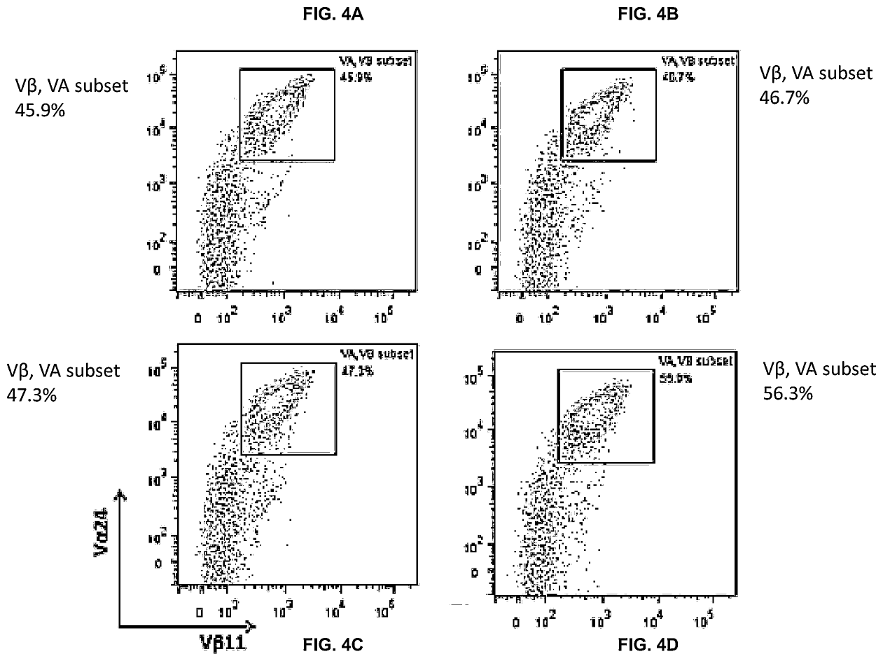

On day 21, 100 μl of the population of CKTCs expanded by processes A, B, C and D were removed, and added into the following fluorescent antibody respectively: TCR Vα-PE and TCR Vβ-FITC. After incubation at 4° C. for 30 min, the proportion of target cells in the cell products was measured by flow cytometry using Forward Scatter (FS) vs 90oLight Scatter bitmap the lymphocyte intact lymphocyte population. Gating (rectangular) on this bitmap, CD56 vs CD3 was measured. Gating on the double positives, Vα24 vs. Vβ11 was measured. As shown in FIG. 4, the proportion of CTKC cells expressing type-I NKT markers of Group D was superior to the other three groups, as earlier addition of IL-12 may have caused the proportion to be lowered. If addition of IL-12 is required, it may be added at a later stage.

### Example 9. Effect of Time of Adding Cytokine IL-12 on the Killing Ability of the Expanded Population of CKTCs

K562 target cells were taken and used to measure the killing ability of the expanded CKTC cells in Group A and Group D in Example 8. The results are shown in Table 4.

TABLE 4
Effect of cytokines
IL-12 on the killing ability of the expanded population of CTKCs

| | Effector:Target Ratio: | | |
|---|---|---|---|
| | 5:1 | 10:1 | 20:1 |
| Group A | 10.36 | 19.78 | 33.92 |
| Group D | 42.19 | 60.57 | 63.71 |

The results show that addition of IL-12 at day 20 (Group D) significantly improved the killing ability of the expanded population of CTKCs.

### Example 10. In Vitro Cytotoxicity on A549 Human Non-Small Cell Lung Cancer Cells

The cytotoxicity of ex vivo expanded and activated CKTCs produced according to methods described herein is characterized against non-small cell lung cancer (NSCLC) targets. Briefly, CKTCs are expanded and activated as set forth in the methods above. A549 (ATCC number CCL-185) NSCLC tumor cells are cultured according to standard growth conditions. A549 cells are collected and re-suspended in PBS at 1×10⁶ cells/mL. A living cell fluorescent dye CMFDA (Life Technologies Corp.) was added at a final concentration of 1 μM, and incubated at 4° C. for 10 minutes. Tumor cells are washed and seeded into 96 well plates at about 1×10⁴ cells/well. CKTC cells are added at a ratio of effector to target of 5:1, 10:1 or 20:1 into the wells which are seeded with the target cells in advance. Each experiment is run in triplicate. After the effector cells and the target cells are co-cultured for 24 hours, the remaining cells in each group are collected and labeled with 7-aminoactinomycin D (7-AAD). After incubation at 4° C. for 10 minutes, the ratio of 7-AAD positive cells to total cells in the labeled target cells was detected by flow cytometry to determine the killing of effector cells to target cells.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for preparing a pharmaceutical composition comprising a cell product containing an expanded and enriched population of superactivated cytokine killer T cells (SCKTCs) comprising, in order:
(a) isolating a population of mononuclear cells (MCs) comprising a population of cytokine killer T cells (CKTCs);
(b) optionally transporting the preparation of (a) to a processing facility under sterile conditions;
(c) culturing the population of MCs in a culture system;
(d) contacting the culture system of step (c) with alpha-galactosylceramide (αGalCer), or an analog or functional equivalent thereof, and with a population of cells comprising CD1d and αGalCer or an analog or functional equivalent thereof, wherein the contacting is sufficient to stimulate expansion of the population of CKTCs;
(e) contacting the culture system of step (d) with IL-2, IL-7, IL-15 and IL-12, in a predetermined order and time of addition, together with a fresh population of cells comprising CD and αGalCer or an analog or functional equivalent thereof, wherein the contacting is sufficient to stimulate activation of some of the population of expanded CTKCs, forming the expanded and enriched population of SCKTCs; and
(f) collecting the expanded and enriched population of SCKTCs from the culture system to form an SCKTC cell product;
wherein the cell product comprising the expanded and enriched population of SCKTCs of (f) is characterized by one or more of an improved ability to secrete effector cytokines or an improved cytotoxicity compared to the population of CKTCs of (a); and
(g) formulating the cell product comprising the expanded and enriched population of SCKTCs of (f) with a pharmaceutically acceptable carrier, to form a pharmaceutical composition comprising the cell product comprising the expanded and enriched population of SCKTCs.

2. The method of claim 1, wherein a source of the mononuclear cells (MCs) in (a) is blood.

3. The method of claim 1, comprising between steps (e) and (f) transporting the culture from the processing facility to a treatment facility.

4. The method of claim 3, wherein the transporting step is initiated within from about 1 hour to about 24 hours after addition of IL12.

5. The method of claim 1, wherein step (c) optionally comprises re-suspending the MCs and adjusting the MCs to a concentration ranging from about 5×10⁵ cells/ml to about 3×10⁶ cells/ml before performing step (d).

6. The method of claim 1, step (e) comprising adding a fresh population of cells comprising CD1d and αGalCer t or an analog or functional equivalent thereof to the culture system.

7. The method of claim 1, wherein the αGalCer, or an analog or functional equivalent thereof is maintained at a constant concentration from step (d) to step (0.

8. The method of claim 7, wherein the concentration of αGalCer, or an analog or functional equivalent thereof, is between about 50 ng/ml to about 500 ng/ml.

9. The method of claim 1, wherein IL-2 is maintained at a constant concentration from step (e) to step (0.

10. The method of claim 9, wherein the concentration of IL-2 ranges from about 10 U/ml to about 100 U/ml.

11. The method of claim 1, wherein the IL-7 is maintained at a constant concentration from step (e) to step (0.

12. The method of claim 11, wherein the concentration of IL-7 ranges from about 20 ng/ml to 200 ng/ml.

13. The method of claim 1, wherein IL-2 and IL-7 are added at about day 7 of culture.

14. The method of claim 1, wherein IL-15 is added at about day 14 of culture.

15. The method of claim 1, wherein the IL-12 is added at about day 20 of culture.

16. The method of claim 1, wherein step (f) is carried out at least about day 21 of culture.

17. The method of claim 1, wherein the IL-15 is maintained at a constant concentration from step (e) to step (0.

18. The method of claim 17, wherein the concentration of IL-15 ranges from about 10 ng/ml to about 100 ng/ml.

19. The method of claim 1, wherein the IL-12 is maintained at a constant concentration from step (e) to step (0.

20. The method of claim 19, wherein the concentration of IL-12 ranges from about 10 ng/ml to about 100 ng/ml.

21. The method of claim 1, further comprising a step of characterizing expression of cell surface markers by the expanded and enriched population of SCKTCs by flow cytometry.

22. The method of claim 21, wherein a subpopulation of the expanded and enriched population of SCKTCs comprises one or more of CD3+Vα24+Vβ11 cells, CD3+Vα24− cells or CD3+CD56+ cells.

23. The method of claim 21, wherein the subpopulation of SCKTCs further comprises Vβ11+ cells.

24. The method of claim 1, wherein the expanded and enriched population of SCKTCs comprises from about 40% to about 60% of the total population of CKTCs.

25. The method of claim 1, wherein IL-2 and IL-7 are added to the culture simultaneously.

26. The method of claim 1, wherein IL-2, IL-7 and IL-15 are added to the culture simultaneously.

27. The method of claim 1, wherein the population of MCs in step (c) comprises from about 5×10⁵ cells/ml to about 3×10⁶ cells/ml.

28. The method of claim 1, wherein the cell comprising CD1 and alpha-galactosylceramide (αGalCer) is an antigen presenting cell.

29. The method of claim 28, wherein the antigen presenting cell is a dendritic cell (DC).

30. The method of claim 29, wherein the dendritic cell is loaded with αGalCer.

31. The method of claim 30, wherein the dendritic cell loaded with αGalCer is derived from the MCs and is an adherent cell.

32. The method of claim 30, wherein the dendritic cell loaded with αGalCer is prepared by a method comprising:
(a) isolating a population of mononuclear cells (MCs);
(b) culturing the population of MCs in a culture system;
(c) contacting the culture system with IL-4 and GM-CSF, wherein the contacting is sufficient to induce differentiation of the MCs into dendritic cells;
(d) contacting the culture system with αGalCer, wherein the contacting is sufficient to load the dendritic cells with αGalCer.

33. The method of claim 32, wherein the concentration of IL-4 is 500 U/ml.

34. The method of claim 32, wherein the concentration of GM-CSF is 50 ng/ml.

35. The method of claim 32, wherein step (d) is carried out from about 5 days to about 7 days after step (b).

36. The method of claim 32, wherein the population of MCs in step (b) comprise from about 1×10⁵ cells/ml to about 5×10⁶ cells/ml.

37. The method of claim 32 wherein steps (b)-(d) are carried out in a culture medium selected from RPMI 1640 medium containing 10% fetal bovine serum or 10% autologous serum.

38. The method of claim 1, further comprising a step of replenishing the culture medium in the culture system every 2 to 3 days.

39. The method of claim 1, wherein the MCs are derived from a human subject.

40. The method of claim 2, wherein the MCs are isolated from whole blood by Ficoll-Paque gradient centrifugation.

41. The method of claim 1, wherein steps (c)-(f) are carried out in a culture medium selected from X-VIVO-15 serum-free medium, RPMI 1640 medium containing 10% fetal bovine serum or 10% autologous serum.

42. A pharmaceutical composition comprising a pharmaceutically acceptable carrier and an enhanced and enriched population of superactivated cytokine killer T cells (SCKTCs) produced by the method of claim 1.

43. The pharmaceutical composition according to claim 42, wherein the enhanced and enriched population of SCKTCs comprises a subpopulation of one or more of CD3+Vα24+Vβ11 cells, CD3+Vα24-, CD3+CD56+ cells.

44. The pharmaceutical composition according to claim 43, wherein the subpopulation further comprises Vβ11+ cells.

45. A pharmaceutical composition comprising a pharmaceutically acceptable carrier and a cell product comprising an expanded, activated and enriched population of superactivated cytokine killer T cells (SCKTCs) derived from a population of cytokine killer T cells (CKTCs), the SCKTCs characterized by two or more of an induced secretion of a cytokine, a stimulated proliferation of the SCKTCs, an improved cytotoxicity of the SCKTCs, and modulated expression of one or more markers on the surface of the SCKTCs, compared to an unstimulated, unactivated cytokine killer T cell control population.

46. The pharmaceutical composition according to claim 45, wherein the cytokine whose expression is modulated is one or more selected from the group consisting of IL-4, IL-5, IL-6, or IL-10 and IFNγ.

47. The pharmaceutical composition according to claim 46, comprising low expression of one or more cytokines selected from the group consisting of IL-4, IL-5, IL-6, and IL-10, and high expression of IFNγ.

48. The pharmaceutical composition according to claim 46, wherein cytokine production by the enriched population of SCKTCs is characterized as, IL-5-, IL-6-, IL-10-, IL-4 low, IFNγ high.

49. The pharmaceutical composition according to claim 48, wherein the amount of IFN-γ produced by the population of SCKTCs is about 5000 pg/ml or greater.

50. The pharmaceutical composition according to claim 48, wherein the amount of IL-4 produced by the population of SCKTCs is less than 5 pg/ml.

51. The pharmaceutical composition according to claim 48, wherein a ratio of IFNγ: IL-4 in culture supernatants is equal to or greater than 1000.

52. The pharmaceutical composition according to claim 45, wherein a killing rate of a target cell by the enriched population of SCKTCs ranges from about 25% to about 75%, inclusive.

53. The pharmaceutical composition according to claim 45, wherein the killing rate of the population of SCKTCs is at least 1.5 fold greater than the killing rate of nonexpanded, nonactivated cytokine killer T cell control cells.

54. The pharmaceutical composition according to claim 45, wherein a ratio of IFN-γ: IL-4 is at least 1000, and the killing rate is increased at least 1.5 fold greater than the killing rate of nonexpanded, nonactivated cytokine killer T cell control cells.

55. The pharmaceutical composition according to claim 45, wherein the expanded and enriched population of SCKTCs comprises a subpopulation of SCKTCs that express NKT cell markers.

56. The pharmaceutical composition according to claim 55, wherein the expanded and enriched population of SCKTCs cells comprises a subpopulation comprising one or more of CD3+Vα24+ cells, CD3+Vα24− cells or CD3+CD56+ cells.

57. The pharmaceutical composition according to claim 55, wherein the expanded and enriched population of SCKTCs comprises a subpopulation of SCKTCs that are CD3+CD56+.

58. The pharmaceutical composition according to claim 55, wherein the expanded and enriched population of SCKTCs comprises a subpopulation of SCKTCs that express type 1 NKT cells markers.

59. The pharmaceutical composition according to claim 58, wherein the type 1-NKT cell markers comprise TCR Vα and TCR Vβ markers.

60. The pharmaceutical composition according to claim 58, wherein the subpopulation of SCKTCs that express type 1 NKT cells markers comprises a population of cells characterized by expression of one or more of markers CD3+Vα24+, CD3+Vα24-, or CD3+CD56+.

61. The pharmaceutical composition according to claim 45, wherein the expanded and enriched population of SCKTCs derived from a population of cytokine killer T cells (CKTCs) constitutes from about 40% to about 60% of the total CKTC population.

62. The pharmaceutical composition according to claim 45, wherein the pharmaceutical composition comprises a stabilizing amount of serum that is effective for retention by the expanded and enriched population of SCKTCs of their T cell effector activity.

63. The pharmaceutical composition according to claim 62, wherein the stabilizing amount of serum is at least 10%.

64. The pharmaceutical composition according to claim 62, wherein the serum is human serum.
